(19) United States
(12) Patent Application Publication
(10) Pub. No.: US 2026/0108275 A1
(43) Pub. Date: Apr. 23, 2026

(54) MOTORIZED STRUT MOTOR FEEDBACK

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Brian Roberts, Germantown, TN (US); Sied W. Janna, Memphis, TN (US); Andrew P. Noblett, Bartlett, TN (US); Johnny R. Mason, Bartlett, TN (US); Scott Smyth, Memphis, TN (US); Paul Bell, Vestavia Hills, AL (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 19/117,776

(22) PCT Filed: Nov. 7, 2023
(86) PCT No.: PCT/US2023/036912

§ 371 Date: Apr. 2, 2025

Related U.S. Application Data
(60) Provisional application No. 63/424,204, filed on Nov. 10, 2022.

(51) Int. Cl. A61B 17/66; A61B 17/00; A61B 17/56; A61B 34/30; A61B 34/32

(52) U.S. Cl. CPC A61B 17/66; A61B 34/32; A61B 2017/00022; A61B 2017/564; A61B 2034/304

(57) ABSTRACT

A spatial frame apparatus for positioning on a patient to treat an injury. The apparatus includes a frame having one or more motorized struts. One or more motorized struts include one or more motors configured to cause movement of one or more motorized struts in one or more directions in accordance with one or more treatment plans. The apparatus includes one or more processing 2024/102351 components communicatively coupled to one or more motorized struts and/or one or more motors. One or more processing components determine one or more loading parameters on the one or more motorized struts. The loading parameters define loading on the one or more motorized struts resulting from the frame being positioned and/or adjusted on the patient. The processing components execute at least one adjustment of one or more motorized struts based on the determined loading parameters.

### CROSS-REFERENCE TO RELATED APPLICATIONS

This is a non-provisional of, and claims the benefit of the filing date of, U.S. provisional patent application No. 63/424,204, filed Nov. 10, 2022, entitled “Motorized Strut Motor Feedback,” the entirety of which is incorporated by reference herein.

### TECHNICAL FIELD

The present disclosure relates generally to orthopedic devices, systems, and methods for facilitating fracture alignment such as the treatment of musculoskeletal conditions with a spatial frame, which includes a motorized strut such as, for example, a geared-motor assembly, configured to provide various data related to motor feedback, load balancing, healing indications, adjustment rates, and other feedback data.

### BACKGROUND

People suffer bone fractures each year. In many instances, a person that suffers a bone fracture is required to use a bone alignment device such as, for example, an external fixation system, a spatial frame, a hexapod, etc. (terms used interchangeably herein without the intent to limit or distinguish) to align two or more bones, bone fragments, bone pieces, etc. (terms used interchangeably herein without the intent to limit or distinguish). Generally speaking, spatial frames allow for polyaxial movement of the coupled bones and are typically used to keep fractured bones stabilized and in alignment during a treatment period.

Generally speaking, the spatial frame includes first and second rings, platforms, frames, bases, etc. (terms used interchangeably herein without the intent to limit or distinguish) intercoupled by a plurality of struts. In use, the struts have adjustable lengths that may be adjusted regularly (e.g., daily) in accordance with a prescription or treatment plan (terms used interchangeably herein without the intent to limit or distinguish). As the lengths of the struts are adjusted, the platforms are controllably manipulated. The treatment plan specifies strut length adjustments to be made over time to ensure successful bone alignment.

Some spatial frames include electronic components, such as, motorized struts, which lengthen and shorten according to a programmed prescription. Motorizing the struts eliminates patient compliance from the system, enables adjustment of frequencies that is not possible with manual struts, and allows for collection of valuable clinical data. However, existing systems are not capable of providing and/or utilizing important accurate feedback data from the spatial frame to optimize a prescription/treatment plan and/or execution thereof.

### SUMMARY

This summary is provided to introduce a selection of concepts in a simplified form that are further described below in the detailed description. This summary is not intended to identify key features or essential features of the claimed subject matter, nor is it intended as an aid in determining the scope of the claimed subject matter.

In one example, the current subject matter relates to a spatial frame apparatus for positioning bone fragments to treat a patient deformity. The apparatus may include a frame having one or more motorized struts. One or more motorized struts may include one or more motors configured to cause movement of one or more motorized struts in one or more directions in accordance with one or more treatment plans. The apparatus may also include one or more processing components communicatively coupled to one or more motorized struts and/or one or more motors. One or more processing components may be configured to determine one or more loading parameters on the one or more motorized struts. The loading parameters may be configured to define loading on the one or more motorized struts resulting from the frame being positioned and/or adjusted on the patient. The processing components may be configured to execute at least one adjustment of one or more motorized struts based on the determined loading parameters.

In any preceding or subsequent examples, the current subject matter may be configured to include one or more of the following optional features. The apparatus may include one or more sensors, gauges and/or any other monitoring components configured to perform monitoring of at least one of the following: loading on one or more motorized struts, current pull by one or more motors from one or more power sources (e.g., batteries) communicatively coupled to one or more motors, and any combination thereof.

In any preceding or subsequent examples, one or more processing components may be configured to determine one or more baseline loading parameters based on at least one action performed by the patient after the frame has been applied to the patient. The baseline loading parameters defining one or more initial loading parameters exerted on the frame and/or one or more motorized struts while the frame is positioned on the patient during performance of the action. One or more loading parameters may include one or more baseline loading parameters.

In any preceding or subsequent examples, one or more loading thresholds may be defined for one or more motorized struts indicating a predetermined amount of loading that may be exerted by the patient on the frame and/or one or more motorized struts while the frame is positioned on the patient during treatment defined in the treatment plan. Upon determining that loading on one or more motorized struts exceeds one or more loading thresholds, one or more motors may be configured to cause one or more motorized struts to execute an adjustment. The adjustment may include at least one of the following: a direction of adjustment of one or more motorized struts, a length of adjustment of one or more motorized struts, an angle of adjustment of one or more motorized struts, a time for adjustment of one or more motorized struts and/or any other parameters for changing positioning of one or more motorized struts, and/or any combination thereof.

In any preceding or subsequent examples, upon determining that loading on one or more motorized struts exceeds one or more loading thresholds, one or more communication components of the apparatus may be configured to transmit a signal to cause execution of such adjustment.

In any preceding or subsequent examples, the apparatus may include one or more machine-learning components. The machine-learning components may be configured to determine expected healing time associated with treatment of the injury to the patient. The machine-learning components may be configured to include one or more machine-learning models that may be trained using one or more historic motor loading data associated with a healing process of injuries. The machine-learning models may be used to predict a time for completion of the healing process and removal of the spatial frame from the patient. In addition to the historic motor loading data, the machine-learning model may be trained using one or more of the following features: patient specific factors, such as, for example, age, weight, bone density, correction area (e.g., anatomy), deformity vs. trauma, comorbidities, and any combination thereof.

In any preceding or subsequent examples, the machine-learning components may be configured to determine one or more adjustment rates for adjusting movement of one or more motorized struts during the treatment process defined in the treatment plan. The machine-learning components may include one or more machine-learning models that may be trained based on the historic motor loading data as related to a healing process of one or more bone injuries. The models may be used to predict one or more adjustment rates and/or changes to one or more adjustment rates. Similarly, for determination of adjustment rates, in addition to the historic motor loading data, the machine-learning model may be trained using one or more of the following features: patient specific factors, such as, for example, age, weight, bone density, correction area (e.g., anatomy), deformity vs. trauma, comorbidities, and any combination thereof.

In any preceding or subsequent examples, each adjustment rate may be associated with a predetermined adjustment rate threshold. The processing components may be configured to modify one or more adjustment rates based on a determination that such predetermined adjustment rate threshold (as associated with a particular adjustment rate) has been exceeded as a result of adjustments performed by one or more motorized struts.

Examples of the present disclosure provide numerous advantages. For example, by providing an ability to obtain and monitor strut loading data (e.g., through current pull of the power source by a motor driving a motorized strut), the current subject matter is capable of providing a more accurate way of monitoring the treatment process, executing any adjustments, as well as determining when healing may be completed. Conventional systems typically suffer from uneven strut loading as well as many other drawbacks and may result in patient pain during adjustment, difficulty in physically adjusting some struts, longer healing times, less than full corrections, etc. The current subject matter addresses these deficiencies by providing an ability to make changes to adjustments of each strut to balance the spatial frame load, thereby potentially reducing patient pain, minimizing the risk of damage to the components of the motorized strut, as well as an improved quality of the healing process to the patient. Moreover, the current subject matter provides the medical professional supervising patient healing time with accurate assessment of the healing process and more information for making a timely decision when spatial frame may be removed. Existing methods of using radiographic evidence to determine healing have been vastly inaccurate, resulting in additional surgeries for the patient, such as, for example, when the frame is removed prematurely. Further, the current subject matter advantageously determines a proper adjustment rate of the struts (and/or changes thereof) throughout treatment. This is in contrast to the existing methods of visual determination of adjustment rates using radiographic images that are frequently inconsistent and/or inadequate.

Further features and advantages of at least some of the examples of the current subject matter, as well as the structure and operation of various examples of the current subject matter, are described in detail below with reference to the accompanying drawings.

### BRIEF DESCRIPTION OF THE DRAWINGS

The accompanying drawings, which are incorporated in and constitute a part of this specification, show certain examples of the subject matter disclosed herein and, together with the description, help explain some of the principles associated with the disclosed implementations. In the drawings, FIG. 1 illustrates a perspective view of a conventional spatial frame including first and second platforms and a plurality of manually adjustable struts coupled thereto;

FIG. 2 illustrates a cross-sectional view of an example of a motorized strut that may be used in a spatial frame such as, for example, within the spatial frame shown in FIG. 1;

FIG. 3A illustrates a perspective view of an alternate conventional spatial frame including first and second platforms and a plurality of manually adjustable struts coupled thereto;

FIG. 3B illustrates a perspective view of an example of a spatial frame including a plurality of geared-motor assemblies coupled to the manually adjustable struts of the spatial frame in FIG. 3A in accordance with one or more features of the present disclosure, the spatial frame including the plurality of geared-motor assemblies and a companion APP to transmit and receive data, instructions, and updates;

FIG. 4 illustrates a side view of a conventional manually adjustable strut;

FIGS. 5A and 5B illustrate various views of an example of a geared-motor assembly in accordance with one or more features of the present disclosure, the geared motor assembly being coupled to a manually adjustable strut;

FIG. 6 illustrates an exemplary control circuit of a spatial frame;

FIG. 7 illustrates an exemplary process for executing a load balancing of a spatial frame positioned on a patient, according to some examples of the current subject matter;

FIG. 8 illustrates an exemplary process for executing a healing process assessment when using a spatial frame positioned on a patient, according to some examples of the current subject matter;

FIGS. 9A-C illustrate exemplary movements of bone segments, according to some examples of the current subject matter;

FIG. 10 illustrates an exemplary process for determining and modifying an adjustment rate of one or more struts in a spatial frame positioned on a patient, according to some examples of the current subject matter;

FIG. 11 illustrates an exemplary computing apparatus, according to some examples of the current subject matter;

FIG. 12 illustrates an example of a storage medium to store spatial frame logic, according to some examples of the current subject matter; and

FIG. 13 illustrates an example computing platform, according to some examples of the current subject matter.

It should be understood that the drawings are not necessarily to scale and that the disclosed examples are sometimes illustrated diagrammatically and/or in partial views. In certain instances, details that are not necessary for an understanding of the disclosed methods and devices or which render other details difficult to perceive may have been omitted. It should be further understood that this disclosure is not limited to the particular examples illustrated herein. In the drawings, like numbers refer to like elements throughout unless otherwise noted.

### DETAILED DESCRIPTION

To address these and potentially other deficiencies of currently available solutions, one or more implementations of the current subject matter relate to methods, systems, articles of manufacture, and the like that can, among other possible advantages, provide a spatial frame having a geared-motor assembly configured to provide various data related to motor feedback, load balancing, healing indications, adjustment rates, and other feedback data.

### I. SPATIAL FRAME

Spatial frames are well known. One known example of a spatial frame is the TAYLOR SPATIAL FRAME® manufactured and sold by Smith Nephew, Inc. As illustrated in FIG. 1 an example spatial frame 100 may form a hexapod having a circular, metal frame with a first platform 102 and a second platform 104 connected by six adjustable length struts 106 (labeled as struts 106-1 through 106-6 in FIG. 1). Each strut 106 may be independently lengthened or shortened relative to the rest of the frame, thereby allowing for six different axes of movement.

Each strut 106 may include an outer body and an inner body, which may be configured as, or be operatively coupled to, a threaded rod (also referred to as a lead screw). The outer body may be coupled to one of the platforms, such as, the second platform 104 by way of a joint as shown. The inner body may be coupled to the other platform, such as, the first platform 102 by way of a joint as shown. To lengthen or shorten one of struts 106, the outer body and the inner body may be moved or translated relative to one another. For example, the strut 106 may include an adjustment nut wherein rotation of the adjustment nut moves the inner body (e.g., threaded rod or lead screw) relative to the outer body to adjust an overall length of the strut.

In use, the spatial frame 100 may be used to treat a variety of skeletal fractures of a patient. Typically, the spatial frame 100 is positioned around the patient and is used to align two or more bone portions. To do so, a length of each strut 106 may be incrementally adjusted (e.g., shortened or lengthened) in accordance with a treatment plan that specifies adjustments to be made to each strut 106 over time to ensure successful bone alignment. In many instances, the length of each strut 106 should be adjusted daily to comply with the provided treatment plan. Adjusting the length of each strut 106 adjusts the distance between the first and second platforms 102, 104, and hence the first and second bone portions coupled thereto.

During use, patient's bones are normally adjusted (e.g., lengthened, shortened, etc.) by adjusting struts manually, for example, by hand or using a wrench to move the bone segments at a rate of approximately 1 mm/day, which is then proceeded by a consolidation phase before the spatial frame is removed.

It is theoretically known to automate and/or motorize adjustment of a spatial frame by motorizing or otherwise automating strut adjustments. For example, one known motorized strut is the Robotic Hexapod System manufactured by Orthospin Ltd. In use, the Robotic Hexapod System includes an offset motor design that engages custom struts positioned between the first and second platforms. That is, the motor includes a spur gear engaged with a second spur gear associated with the threaded rod of the strut. In use, rotation of the motor drives rotation of the threaded rod via the interaction between the spur gears. The Robotic Hexapod System however suffers from a number of disadvantages including being very bulky and having trailing cables.

However, currently commercially available spatial frames are dependent on manual adjustment of each strut. As a result of the requirement for manual adjustments, generally speaking, successful treatment requires patient compliance (e.g., daily manual adjustments to each of the struts) to avoid human error. In routine clinical practice, the treatment plan may require multiple daily adjustments to be made to each of the plurality of struts. For example, a patient may be required to manually adjust one or more of the struts, typically two or more times each day, and often over long periods of time with support from either a family member, a clinician, or both. As such, compliance with the treatment plan may be burdensome, painful, etc.

As a result, the number of adjustments dictated by the treatment plan may be limited. For example, generally speaking, treatment plans often limit the required number of daily adjustments to each of the plurality of struts to four per day. During a normal treatment plan, this may equate to approximately 720 adjustments (e.g., turns) over a 1-month treatment span (e.g., 6 struts×4 adjustments per day×30 days). During an extended treatment plan for more severe applications, this may equate to approximately 2,160 adjustments (e.g., turns) over a 3-month treatment span (e.g., 6 struts×4 adjustments per day×90 days).

In addition, during the treatment period, the patient may require numerous clinical visits to confirm proper strut adjustments to ensure compliance and avoid incorrect adjustment, which has historically been the leading cause of treatment failure.

Motorized and/or automated struts could provide numerous advantages over manually adjustable struts. In use, electric motors, motor-drive units, and a control unit (e.g., a central control unit) could function to supersede the manual actuation of the strut adjustments. For example, a motorized system could eliminate the need for patient compliance and decrease the frequency of post-operative visits for patient supervision given that the spatial frame only has to be activated at the start of the distraction phase and terminated at the end of the distraction phase without any patient intervention. Additionally, automatic distraction could enable a higher distraction frequency and result in smaller excursions per activation. Smaller distraction steps or adjustments have the potential to result in less damage to the distracted tissues, improving bone regeneration and adaptation of the surrounding soft tissues. That is, spatial frames equipped with motorized and/or automated struts offer the potential to increase the number of daily distraction adjustments by enabling finer (e.g., smaller) adjustments at a controllable rate and frequency of distraction that encourages better quality bone formation. Making finer (e.g., smaller) adjustments during limb lengthening can have significant advantages in terms of reduced soft tissue damage, less pain, and opioid usage and accelerated bone healing. One study has found that the bone fixation index was only 5-6 days/cm when using motorized and/or automated distraction compared to 22-24 days/cm by manual adjustment.

In some examples, for example, a motorized strut could be programmed to perform anywhere from 1 adjustment per day to continuous adjustments. In some examples, finer adjustments can increase the number of adjustments over a 1-month period from approximately 720 adjustments to approximately 3,600 adjustments (e.g., 6 struts×20 adjustments per day x 30 days). In another example, finer adjustments can increase the number of adjustments over a 1-month period to approximately 259,200 adjustments (e.g., 6 struts×1440 adjustments per day×30 days). Over an extended 3-month treatment period, this could increase the number of adjustments from approximately 2,160 adjustments to approximately 10,800 adjustments (e.g., 6 struts×20 adjustments per day×90 days). In another example, finer adjustments can increase the number of adjustments over a 3-month period to approximately 777,600 adjustments (e.g., 6 struts×1440 adjustments per day×90 days).

In use, each motorized strut may include a motor and may be used in a spatial frame such as, for example, spatial frame 100, to move the first and second platforms 102, 104, respectively, to align two or more bone portions. In use, the spatial frame and/or system architecture may be arranged and configured to automatically adjust the motorized struts according to the prescribed treatment plan (e.g., automatically adjust the plurality of motorized struts without patient intervention). Alternatively, the spatial frame and/or system architecture may be arranged and configured to require patient and/or caregiver activation to begin the process of automatically adjusting the motorized struts according to the prescribed treatment plan. For example, the spatial frame may be arranged to intermittently auto-adjust the motorized struts at predetermined times according to the treatment plan. Alternatively, the spatial frame may be arranged to intermittently auto-adjust the motorized struts at select times when convenient and/or selected by the patient. Alternatively, the spatial frame may be arranged and configured to continuously auto-adjust the motorized struts in small discrete increments.

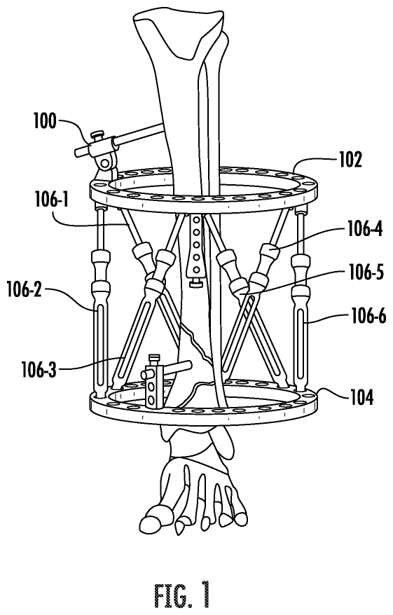

Referring to FIG. 2, an example of a motorized strut 200 is shown. In use, the motorized strut 200 may be coupled to first and second platforms in a spatial frame. For example, the motorized strut 200 may be used in place of the manually adjustable struts 106 shown in FIG. 1. As shown in FIG. 2, the motorized strut 200 may include an outer body 202 operatively coupled with a first joint 204 for coupling to a first platform, an inner body 210 operatively coupled with a second joint 212 for coupling to a second platform, and a drive mechanism, actuator, etc. 220 (used interchangeably herein without the intent to limit or distinguish). In use, actuation of the drive mechanism 220 moves the inner body 210 relative to the outer body 202 to adjust a length of the motorized strut 200.

As illustrated, the drive mechanism 220 may include a motor 222 and a threaded rod or lead screw 224 arranged and configured so that, in use, actuation of the motor 222 rotates the threaded rod 224, which moves the inner body 210 relative to the outer body 202 to adjust an overall length of the motorized strut 200. In addition, the drive mechanism 220 may include one or more gears to adjust speed and torque of the motor 222.

In addition, the motorized strut 200 may include any required circuity. For example, the motorized strut 200 may include one or more position sensors to, for example, monitor absolute position or length of the motorized strut 200. In addition, and/or alternatively, the motorized strut 200 may include other sensors for monitoring various biomechanical parameters such as, for example, a force sensor 230 for monitoring stresses and forces, across the bone gap and/or the soft tissues (muscle, apposing cartilage or peripheral sensory nerves), an accelerometer for capturing patient ambulation data (steps, distance, speed and cadence), a gyroscope for measuring the degree of alignment between the bone fragments, and a sensor motor support 232, etc. In addition, and/or alternatively, the motorized strut 200 may include an encoder such as, for example, a rotary encoder for measuring rotation of the motor 222 for accurate positioning and motion control. In addition, and/or alternatively, the motorized strut 200 may include flash memory for storing unique identifiers (e.g., addresses) and for storing current position, biomechanical and ambulatory data, etc.

As illustrated, the motorized strut 200 may be arranged and configured with an in-line design, wherein the motor 222 shares a common longitudinal axis as the threaded rod 224 and the telescoping portion (e.g., inner body 210) (e.g., the motor and electronics are housed in an enclosure or body that shares the same axis as the threaded rod, adjustment nut, and telescoping portion of the strut).

Additional information on examples of motorized spatial frames can be found in International Patent Application No. PCT/US20/52276, filed on Sep. 23, 2020, entitled “Automated Spatial Frame and Automated Struts Used Therewith,” the entire contents of said application being hereby incorporated by reference in its entirety herein.

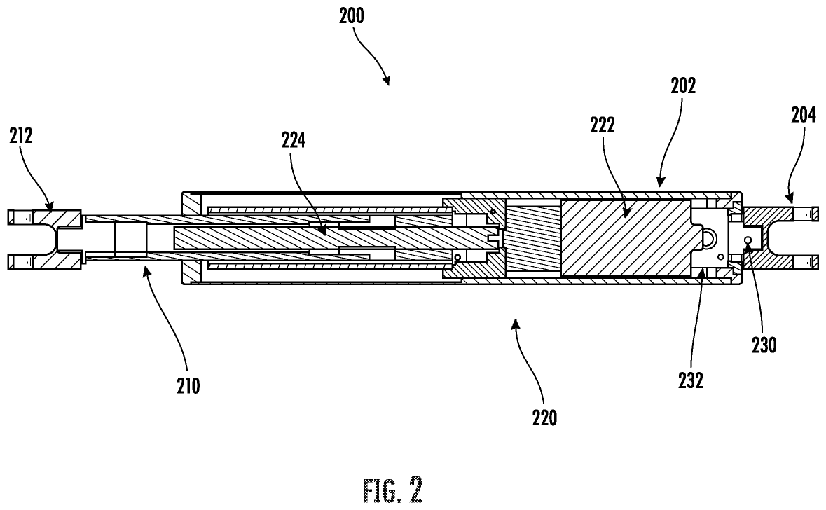

With reference to FIGS. 3A-5B, in some examples, the motorized strut may be a geared-motor assembly. The geared-motor assembly may be arranged and configured as a self-contained unit arranged and configured to receive and transmit data with an external computing system. The geared-motor assembly including an enclosure or housing containing a motor, a power supply, a microprocessor, and all other power and control circuity needed to engage and control a manually adjustable strut in a spatial frame.

In some examples, each geared-motor assembly may include a motor and a torque transmitting mechanism such as, for example, a gear, arranged and configured to engage a corresponding gear on a manually adjustable strut in a spatial frame. In use, actuation of the motor enables motorized rotation of the torque transmitting mechanism and thus the manually-adjustable strut coupled to the geared-motor assembly. In addition, each geared-motor assembly may include a microcontroller arranged and configured to control operation of the geared-motor assembly including, for example, receiving and/or updating a treatment plan and/or controlling activation of the motor without the need for a separate centralized master control unit positioned within the spatial frame. Each geared-motor assembly may further include a wireless communication chip or antenna arranged and configured to communicate with an external computing system to, for example, exchange data relating to strut position, exchange data relating to and updating the prescribed treatment plan, etc. Each geared-motor assembly may also include a power supply such as, for example, batteries, to power the geared-motor assembly including, for example, the motor, the microcontroller, the wireless communication chip, and any associated sensors and/or additional circuity. Each geared-motor assembly may also include a sensor for either positional control, biomechanical feedback, or a fault level detection in the gear train. In some examples, each geared-motor assembly may include its own self-contained power management, wireless communication, and microcontroller unit that controls the position of the strut.

In some examples, the geared-motor assemblies are arranged and configured to be used in a spatial frame. The spatial frame includes a plurality of manually adjustable struts coupled to first and second platforms. Movement of the struts move the first and second platforms, and hence the first and second bone portions coupled thereto. FIG. 3A illustrates an example of a spatial frame 300. The spatial frame 300 includes a first platform such as, for example, first platform 102, a second platform such as, for example, second platform 104, and a plurality of manually adjustable struts such as, for example, struts 106, coupled to the first and second platforms 102, 104.

Each of the geared-motor assemblies may be coupled to one of the manually adjustable struts. Thus arranged, the spatial frame can be selectively configured to operate in either of a first or manually adjustable mode or configuration of operation wherein each strut may be manually adjusted or a second or motorized mode or configuration of operation wherein a geared-motor assembly may be coupled to each strut to facilitate motorized adjustment of the struts. In addition, by utilizing a geared-motor assembly that couples to a manually adjustable strut by, for example, interconnecting corresponding gears, an offset motor design is achieved thereby enabling a shorter minimum strut length to be achieved (e.g., the geared-motor assemblies may be arranged and configured with a shorter minimum length (e.g., length of the strut as measured end to end (e.g., joint to joint) with the threaded rod assembly in the fully retracted position)) as compared to conventional in-line motorized struts, while still providing a reasonable working length (e.g., adjustment length of the strut in use—length adjustment or difference between the minimum length and the maximum length of the strut).

As shown in FIG. 3B, during use, the removable geared-motor assembly 302 is arranged and configured to engage, attach, couple, etc. to the manually adjustable struts 106 of the spatial frame 300. Thus arranged, the spatial frame 300 can be operated in and switched between two modes or configurations of operation. In the first mode or configuration of operation, the struts 106 may be manually adjustable, as illustrated in FIG. 3A. In the second mode or configuration of operation, a geared-motor assembly 302 may be attached to one or more of the manually adjustable struts 106 to enable motorized and/or automated adjustment of the struts.

The geared-motor assemblies 302 are coupled to the manually adjustable struts 106 of the spatial frame 300. In some examples, the geared-motor assemblies 302 may be coupled to the manually adjustable struts 106 after surgery in clinic by, for example, a primary care provider. Alternatively, or in addition to, the geared-motor assemblies 302 may be coupled to the manually adjustable struts 106 at any time and by anyone. Once coupled, the geared-motor assemblies 302 may facilitate motorized and/or automated adjustments such as, for example, semi-continuous actuation. In some examples, the geared-motor assemblies 302 may enable motorized adjustments to be made autonomously via a companion APP running on, for example, a smartphone, a tablet, or other external computing system. Thus arranged, the spatial frame and/or system architecture may be arranged and configured to automatically adjust the motorized struts according to the prescribed treatment plan (e.g., automatically adjust the plurality of struts without patient intervention). Alternatively, or in addition to, the spatial frame and/or system architecture may be arranged and configured to require patient and/or caregiver activation to begin the process of automatically adjusting the struts according to the prescribed treatment plan. For example, the spatial frame may be arranged to intermittently auto-adjust the motorized struts at predetermined times according to the treatment plan. Alternatively, or in addition to, the spatial frame may be arranged to intermittently auto-adjust the motorized struts at selected times when convenient and/or when selected by the patient.

In some examples, the geared-motor assemblies 302 may each include an enclosure or housing 510, a coupling mechanism 520 for coupling the geared-motor assembly 302 to the strut 106, a motor 530, a torque transferring mechanism 531 (e.g., a transmission or gears for transferring rotation from the motor 530 to the strut 106), and all necessary components and circuity so that activation of the motor 530 moves the strut 106. For example, the gear-motor assemblies 302 may include one or more microprocessors, sensors such as, for example, positional sensors to monitor the length of the struts, load sensors or accelerometer for providing biomechanical feedback during bone healing and acoustic emission or vibration sensor for fault level detection in the gear train, a communication chip or antenna for facilitating communication and/or transfer of data, a power supply such as, for example, a battery, a charging circuit, etc.

Thus arranged, by utilizing geared-motor assemblies 302, motorized and/or automated adjustments of a spatial frame can be achieved. In use, the geared-motor assemblies 302 are arranged and configured to engage a manually adjustable strut 106 in an outpatient setting thus enabling the spatial frame to be operated in two different modes or configurations: (a) a standard, manual adjustment mode where the lengths of the struts 106 can be adjusted by manual rotation of a threaded adjustment nut and (b) motorized and/or automated adjustment via the geared-motor assemblies 302.

In some examples, by arranging the geared-motor assemblies 302 as self-contained units or devices incorporating wireless, self-powered, and incorporating their own microprocessors (e.g., in some examples, the geared-motor assemblies 302 are arranged and configured as a self-contained unit including all of the necessary components and circuity to control each strut according to the prescribed treatment plan), the geared-motor assemblies eliminate the need for any external cables or wires that could snag during use and eliminate the need for incorporating a centralized master control unit onto one of the platforms of the spatial frame thereby reducing bulk and safety risk to the patient (e.g., self-containment of the control circuitry, wireless communication chip, and power source within geared-motor assemblies negate the need for cables and a centralized master control unit positioned elsewhere on the spatial frame along with any needed cables or wires).

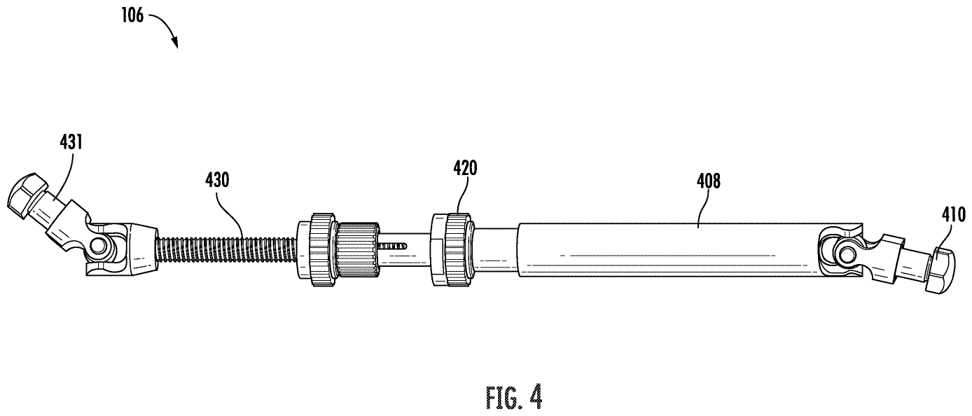

In addition, by utilizing geared-motor assemblies, existing features of the manually adjustable struts are retained. That is, with the geared-motor assemblies detached from the manually adjustable struts, operation of the struts is unaffected. For example, if the manually adjustable strut incorporates a quick adjustment feature (e.g., quick adjustment nut 508 shown in FIG. 5B) to enable manual lengthening of the strut without rotating the threaded nut or rod, such adjustment feature is retained thus enabling faster adjustment during, for example, initial setup in the operating room. Moreover, the geared-motor assemblies provide an offset motor design allowing greater application or use. For example, by incorporating an offset motor design, a shorter minimum strut length can be achieved (approximately 80 mm), which allows the struts to be used for correcting deformities in, for example, children with shorter limbs.

In some examples, when arranged in a spatial frame, the geared-motor assemblies may be arranged and configured to wirelessly exchange data, instructions, etc. with an external computing system such as, for example, a smartphone, a tablet, a computer, etc. running a companion APP. However, it is envisioned that the geared-motor assemblies may exchange data with an external computing system by any now known or hereafter developed system. For example, each of the geared-motor assemblies may include a communication interface to exchange data over a wired connection.

In some examples, the geared-motor assemblies may be water-proofed to facilitate the patient, for example, taking a shower or bath. Alternatively, or in addition to, it is envisioned that the geared-motor assemblies could be removed prior to showering and/or the spatial frame may be covered by, for example, a bag during a shower thus alleviating the necessity for water-proofing each of the geared-motor assemblies. The geared-motor assembly may also eliminate the need for sterilization since the geared-motor assemblies can be coupled to the struts in clinic.

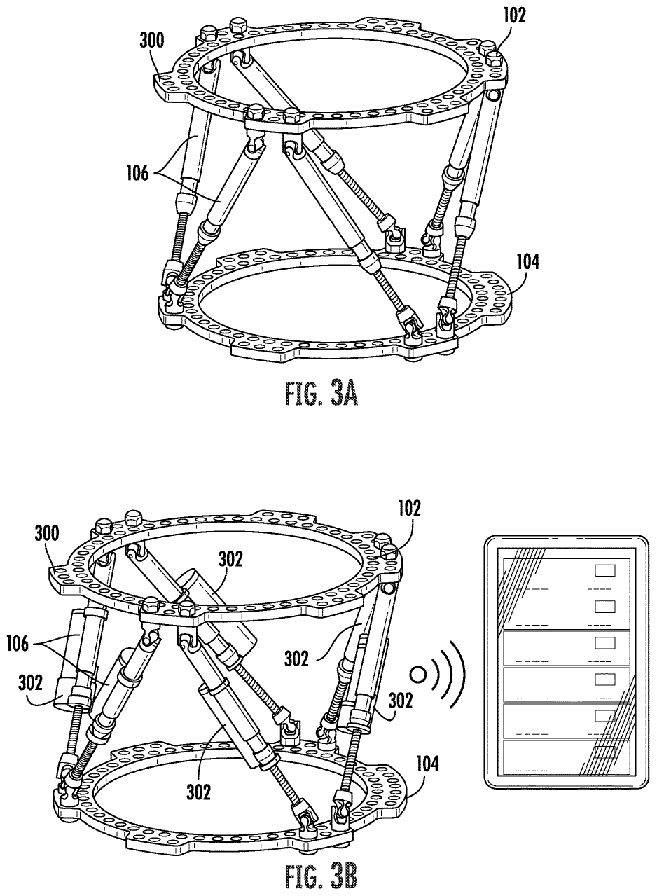

FIG. 4 shows a conventional manually adjustable strut, such as, for example, strut 106. As will be readily appreciated by one of ordinary skill in the art, the manually adjustable strut 106 includes an outer body 408 including a first joint 410 for coupling to a first platform, an internally threaded member or adjustment nut 420 coupled to the outer body 408, and an externally threaded rod or lead screw 430 including a second joint 431 for coupling to a second platform, the externally threaded rod 430 threadably engaging the adjustment nut 420. In use, the externally threaded rod 430 is constrained such that it cannot rotate relative to the outer body 408, the adjustment nut 420 is rotatably coupled to the outer body 408 but cannot translate. Thus arranged, in use, rotation of the adjustment nut 420 causes the externally threaded rod 430 to move (e.g., translate) relative to the outer body 408 to lengthen or shorten the length of the strut 106 depending on the direction of rotation. During use, the adjustment nut 420 can be manually rotated, for example, by hand or using a wrench.

In some examples, by coupling a motor to the strut 106, motorized and/or automated adjustment of the strut 106 can be achieved. For example, with reference to FIGS. 5A and 5B, in some examples, the adjustment nut 420 could be modified to include teeth into an outer diameter thereof. Alternatively, or in addition to, the adjustment nut 420 could be replaced with a gear or the strut 106 could be modified to include a gear coupled to the threaded rod 430. In any event, in use, an interface is created for coupling the motor of the geared-motor assembly to the strut 106.

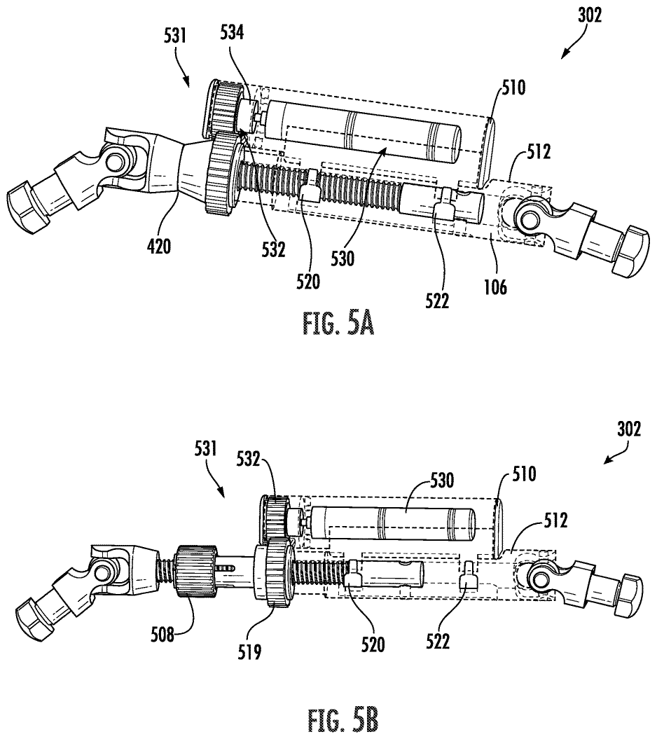

Referring to FIGS. 5A and 5B, the geared-motor assemblies 302 include a housing or enclosure 510 (terms used interchangeably herein within the intent to limit or distinguish). In use, the housing 510 is arranged and configured to enclose, or at least partially enclose, all of the components of the geared-motor assembly 302. For example, in some examples, with additional reference to FIG. 6, the geared-motor assembly 302 includes a control circuit 650 (e.g., a printed-circuit board (PCB)), a microcontroller 652, a wireless communication chip, a power supply 654 such as, for example, one or more batteries, and a charging circuit 656. The electronics and the power source being housed with the motor 530 inside the housing 510. In use, when properly coupled to each of the struts 106, the geared-motor assemblies 302 facilitate motorized and/or automated adjustment of the strut 106. In addition, the geared-motor assemblies 302 may be coupled (e.g., wirelessly coupled) to an external computing system running, for example, a companion application.

In use, the geared-motor assembly 302 can be mounted to the manual struts 106 via a coupling mechanism 520, which can be arranged in any suitable mechanism now known or hereafter developed to couple or mount the geared-motor assemblies 302 to the struts 106 including, for example, clips, sleeves, magnets, straps, etc. In some examples, the coupling mechanism 520 enables easy attachment and detachment of the geared-motor assembly 302 from the strut 106 to facilitate a change in mode between manual and automated adjustment. In some examples, as shown in FIGS. 5A and 5B, the geared-motor assembly 302 may include spring loaded arms 522 arranged and configured to enable the geared-motor assembly to clip onto or engage the outer body 408 of the strut 106. In some examples, in order to better accommodate the geared-motor assembly 302, the outer body 408 of the strut 106 may be modified to include a flat surface and/or grooves 512 formed in the outer surface thereof. Thus arranged, by modifying a conventional strut to include a gear or gear teeth and optionally modifying the strut to include one or more flats and/or grooves for accommodating the geared-motor assembly 302, the modified conventional strut could be used for both manual and motorized adjustment cases.

As previously mentioned, in some examples, the geared-motor assembly 302 includes a torque transferring mechanism 531 for transferring torque from the motor 530 to the strut 106. In use, the torque transferring mechanism 531 can be any suitable mechanism now known or hereafter developed. For example, as previously described, the strut 106 and the motor 530 may include first and second gears 519, 532, respectively. In use, the first gear 519 is operatively associated with the strut 106. The second gear 532 is operatively associated with the motor 530 so that activation of the motor 530 drives (e.g., rotates) the second gear 532, which rotates the first gear 519 thereby translating the threaded rod 430 of the strut 106.

The first and second gears 519, 532 may be any suitable gear now known or hereafter developed. For example, the first and second gears 519, 532 may be pinon gears, spur gears, helical gears, a worm gear mechanism (as will be described in greater detail below), etc. Alternatively, the torque transferring mechanism 531 may be a belt drive system. In some examples, the adjustment nut 420 of the strut 106 can be modified to include an external-toothed geared surface thereby transitioning the adjustment nut 420 of the strut 106 into the first gear 519 so that the adjustment nut 420 of the strut 106 can be directly driven by the gear 532 attached to the output shaft 534 of the motor 530. Alternatively, or in addition to, the strut 106 can include a gear (e.g., the first gear 519). For example, in some examples, the first gear 519 can be mounted on the threaded rod 430. In some examples, the first gear 519 can be mounted on the threaded rod 430 as a compression-fitted collar. In use, the first gear 519 engages with the second gear 532 located on the output shaft 534 of the motor 530. Thus arranged, activation of the motor 530 rotates the second gear 532, which rotates the first gear 519, which causes the strut 106 to move.

In some examples, and as previously mentioned, the geared-motor assembly 302 may include a control circuit 650. The control circuit 650 may be arranged and configured to enable autonomous, ultra-low speed movement of the strut (0.002 mm/s) and a survey of the mechanical loads exerted on the motor 530 through computation of the motor torque (DC motor current correlates with torque load on motor).

Referring to FIG. 6, as described above, the geared-motor assembly 302 includes a control circuit 650 arranged and configured as a control board or print-circuit board (PCB). As illustrated, the PCB may include a microcontroller 652, a wireless communication chip, a power supply 654 such as, for example, one or more batteries (e.g., coin cells), a charging circuit 656, and any other circuity or components needed to operate the geared-motor assemblies 302 as described herein including, for example, various surface mount devices (SMD), diodes, resistors, inductors, capacitors, etc. The control circuit 650 may be housed within the housing 510 adjacent to and extends (or runs) substantially parallel to the motor 530.

Additional information on examples of geared-motor assembly can be found in U.S. patent application Ser. No. 63/312,760, filed on Feb. 22, 2022, entitled “Detachable Geared-Motor Assembly for Motorizing a Strut in a Spatial Frame,” the entire contents of said application being hereby incorporated by reference in its entirety herein.

It should be appreciated that while various examples of a motorized strut have been disclosed herein, it should be appreciated that one or more features of the present disclosure can be used with any suitable motorized struts now known or hereafter developed. As such, the present application should not be limited to any particular configuration or type of motorized strut unless explicitly claimed.

### II. MOTORIZED STRUT FEEDBACK

### A. Using Strut Motor Feedback to Determine Load Balancing

As described above, spatial frames or hexapods are used to treat deformity correction and traumatic injuries. This is accomplished using six struts that are adjusted daily to move bone segments into a desired position. As bone segments move, the process of distraction osteogenesis takes place and bone is grown by the body to bridge the gap between the segments. During this process, in conventional systems, the struts might not be loaded evenly throughout the treatment process as adjustments are made (e.g., one or two struts may carry more load than any other struts). This can be due to many factors including, for example, but not limited to, a fixation placement, a correction path that must be followed to complete limb alignment, even potential partial bone healing, and others.

However, existing solutions suffer from uneven strut loading during treatment and fail to address inconsistencies associated therewith. When struts become difficult to adjust by hand, patients use a 10 mm wrench to adjust them, but often these difficult adjustments are associated with higher-than-average patient pain. When patients report high pain with adjustments, they may be told to pause their adjustments for a time to let the pain subside before performing another adjustment. This may lead to incorrect healing of the bones, which may result in longer healing period, surgeries, and other complications.

In some examples, the current subject matter relates to an ability to monitor adjustments and thus, address uneven strut loading. The current subject matter may be configured to monitor strut loading by monitoring motor current pull as the struts are actuated. Based on the data obtained during monitoring, the current subject matter may be configured to correlate how much current the motor pulls when making an adjustment with how much load each strut is experiencing. Based on the loading on each strut as correlated with motor load, the current subject matter may be configured to use the motor load to monitor and change loading on each strut, accordingly.

As discussed above in connection with FIGS. 5A-6, each strut of the motorized spatial frame may be configured to include a PCB, a battery, and a motor. As can be understood, each of and/or some and/or all the struts may include all three of these components and/or some of these components. For instance, one or more batteries and/or PCB components may be housed on the ring of the motorized spatial frame and/or anywhere else on the spatial frame. The battery may be configured to provide power to the motor. The motor may cause lengthening and/or shortening of one or more struts by turning one or more gears that interact with an internally threaded member coupled to the threaded rod of the strut. As the strut is loaded with soft tissue loads and/or weight bearing loads, the amount of torque required from the motor to overcome the resistance of the loads in the system will vary. As the torque required varies, the current pulled from the battery by the motor may also vary in a corresponding manner.

In an ideal scenario, each strut may be loaded evenly throughout treatment to, for example, impart the least possible stress on the battery and/or motor, however, this is not possible due to the uniqueness of each patient's body geometry, body weight, soft tissue forces, activity level, placement of the spatial frame, characteristics of the spatial frame, and/or any other factors that may affect strut loading. In view of the variability of the loading, the current subject matter may be configured to establish a baseline loading for each frame at the beginning of the adjustment phase of treatment.

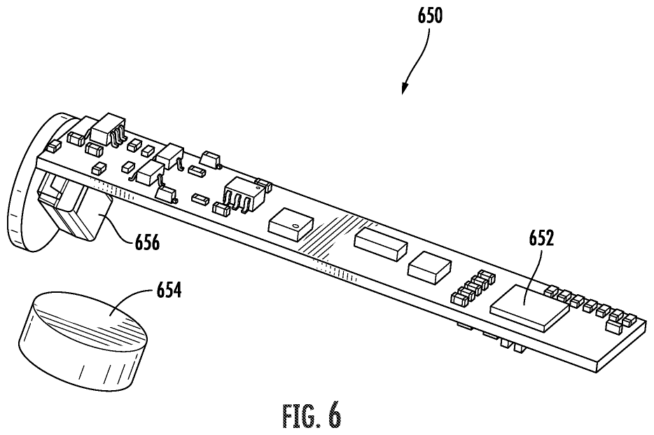

FIG. 7 illustrates an exemplary process 700 for executing a load balancing of a spatial frame positioned on a patient, according to some examples of the current subject matter. The process 700 may be executed using a spatial frame shown in FIGS. 3a-6. In particular, the spatial frame may be communicatively coupled to one or more user devices (e.g., a mobile telephone, a tablet, a laptop, a personal computer, a personal digital assistant, and/or any other device) via one or more networks. The connection may be wireless and/or wired and/or any combination thereof.

The spatial frame may be configured to incorporate one or more transceivers (e.g., wireless, wired, etc. transceivers) that may be configured to transmit and/or receive data from the spatial frame. The data may include current pull data corresponding to an amount of current drain from the battery and/or batteries that may be used as power source(s) by one or more motors used to perform movement of each and/or one or more motorized struts 106. The data may also include loading data (e.g., pressure, tension, etc.) exerted on each motorized strut 106 by the patient body, limbs, etc. during sitting, standing, performing one or more activities, etc.

In some examples, the spatial frame may also incorporate one or more sensors that may be configured for measurement of strut loads, current pull loads, etc. By way of a non-limiting example, a sensor may be positioned on each strut for detecting strut load. Each such sensor may be configured to provide data to the transceiver for transmission to an external device for further processing (e.g., calculation of the load). Alternatively, or in addition, each such sensor may be configured to transmit its data directly to the external device. Further, a single sensor may be configured to monitor loading data for all struts individually and/or collectively and then provide its data to the frame's transceiver and/or directly to the external device. Alternatively, or in addition, a single sensor may be configured to monitor loading data for a group of struts, while other sensor(s) may be configured to monitor loading data for another group of struts, where each such sensor may be configured to report its monitored data to the transceiver and/or external device. The sensors may be configured to be communicatively coupled to one another and/or the transceiver and/or external device using a wireless and/or a wired connection and/or any combination thereof.

Alternatively, or in addition, the spatial frame may be configured to include processing, storage and/or communication capabilities for controlling actuation of each of the struts in the spatial frame, receiving/obtaining current loading data from the motor as a result of actuation of the struts and/or loading data from each of the struts, determining loading on each of the struts, and further controlling adjustments of each strut as a result of the determined loading. Such processing, storage and/or communication capabilities may be incorporated into the PCB of the spatial frame and/or be separately positioned anywhere on the frame.

In some examples, the spatial frame may be used in connection with one or more treatment plans that may be designed for the spatial frame, the patient, and/or the specific injury, deformity, etc. that the spatial frame may be intended to treat. The treatment plan(s) may be designed by a medical professional (e.g., a surgeon, a doctor, etc.) that may use one or more software-based programs to aid in creation of such plan(s). The plan(s) may define one or more load thresholds on one or more struts and/or one or more motors. The plan(s) may also specify one or more responses/reactions (or expected responses/reactions) by the strut(s) and/or motor(s).

Once the treatment plan(s) is defined, it may be uploaded to one or more memory and/or one or more storage locations communicatively coupled to one or more processors/processing component(s) of the frame for execution. As can be understood, the frame may include a single processor that may control operation of all struts, and/or one or more processors that may be controlling operation of one or more struts (e.g., one processor controls operation of one strut, and/or one processor controls operations of multiple struts, etc.).

The processor(s) may then begin execution of the uploaded treatment plan(s) triggering motor(s) to cause movement(s) by the respective strut(s). The movement(s) may continue until a determination is made by one or more processors that one or more predetermined threshold(s) associated with loading on one or more struts has been exceeded and/or about to be exceeded. The processors associated with struts may be configured to communicate with one another to determine when predetermined threshold(s) have been exceeded and/or about to be exceeded. Upon determination that predetermined threshold(s) have been exceeded and/or about to be exceeded, the processor(s) may be configured (using programs that may be uploaded together with treatment plan(s)) to respond/react by changing adjustments, rates of adjustments, etc. in accordance with the uploaded treatment plan(s). The processor(s) may further continuously monitor each strut (e.g., its operation, loading, etc.) to perform any adjustments dynamically (e.g., as they occur and/or about to occur, and/or “on the fly”).

Referring to FIG. 7, at 702, a prescribed treatment plan associated with treatment of a patient using a spatial frame (e.g., shown in FIGS. 3a-6) may be received. The treatment plan may be configured to indicate how the spatial frame needs to be adjusted during the course of the treatment and when.

At 704, one or more frame loading baseline parameters and/or a baseline may be established for the spatial frame in accordance with the received treatment plan. To establish the baseline, the spatial frame may be positioned on the patient, at 701. The current subject matter may be configured to actuate each/some/all struts for a predetermined period of time while the patient is directed to stand, sit, walk and/or perform any other activity, at 703. As a result of these activities, the current subject matter may be configured to determine loading of the motor, at 705 and, using the motor loading data, determine loading for each strut in the spatial frame, at 707. This data may be used to establish the loading baseline thereby providing an understanding of the strut loads associated with soft tissue forces versus weight bearing forces during daily activities (and/or any other type of activities). With the loading baseline established, the current subject matter may be configured to monitor for changes throughout the course of treatment, at 706. In some examples, the loading on each strut (and/or differences in loading vis-a-vis the loading baseline) may be monitored and compared to a predetermined threshold to ascertain changes, at 708. If no changes are detected, then the current subject matter may be configured to continue performing strut adjustments in accordance with a prescribed treatment plan, at 711, as well as continue monitoring of loading on each strut of the spatial frame, at 706.

The orientation of the frame on the patient and the direction of the correction determined by the treatment plan may often result in some struts being loaded significantly more than others. For example, loads experienced on some struts may make prescribed adjustments more difficult. Further, counteracting the increased strut loads may require additional power be applied to the motors associated with the more heavily-loaded struts. Additionally, strut loads (and/or overloading of one or more struts) may make adjustments by the motors nearly impossible.

If, at 708, significant strut load imbalance is detected, the current subject matter (e.g., a PCB/processor) may be configured to attempt performing adjustments through off-loading of overloaded struts (e.g., using one or more adjustment protocols), at 709, such as, for example, by transmitting one or more signal(s), at 710, to the motors to initiate one or more adjustments (e.g., small adjustments) of some of the struts, at 712, so as to off-load the struts experiencing higher loads. As part of this process, the spatial frame's PCB/processor may be configured to perform an evaluation of loading on one or more of the struts (e.g., those that have been determined to be overloaded) and determine whether or not the overloaded strut has or has not been off-loaded. In some examples, the current subject matter may be configured to implement one or more software control mechanisms to ensure that the struts being adjusted are only adjusted in the manner that does not significantly deviate from the prescribed treatment plan.

At 714, a determination may be made whether or not the overloaded strut has or has not been off-loaded. If it has been off-loaded, the processing may return to 706 to continue monitoring of loading on each strut in accordance with the prescribed treatment plan. Otherwise, when problematic uneven strut loading is detected, at 714, the current subject matter may be configured to execute one or more other adjustment protocols, at 716. For example, in most cases, all motorized struts may be configured to be adjusted nearly simultaneously in accordance with the treatment plan. However, when excessive loads on one or more struts are detected, the current subject matter may be configured to select an adjustment protocol to attempt to balance strut loads, such as, for example, by slowing a rate of adjustment on one or more struts experiencing excessive loads and allowing adjacent (and/or any other struts), e.g., more lightly loaded, struts to adjust by a limited magnitude, according to the prescribed treatment plan. As strut adjustments are slowed, the current subject matter may be configured to continue monitoring current loads pulled by all motors to determine whether these changes are effective. If the overloaded struts are effectively off-loaded, then all struts may once again adjust simultaneously according to the treatment plan. Strut load conditions may be continuously monitored so that the process may be repeated each time a strut becomes overloaded. If one or more overloaded struts cannot be off-loaded after execution of one or more adjustment protocols, at 718, the patient may be directed to consult with the healthcare professional supervising patient's treatment, at 720.

Another adjustment protocol may include modifying one or more ending values for one or more struts for one or more days until strut loads are balanced within a predetermined range. This allows for a dynamic prescription treatment plan that may be configured to automatically make changes to adjustment of the struts in real time so that strut loads are more balanced but still adjusting within a controlled threshold of the treatment plan. This may, in some examples, include changing the number of days of the correction from the number identified in the initial treatment plan. If the loads cannot be balanced and continue to increase on overloaded struts, further changes may need to be implemented (e.g., the patient may need to consult with the treatment doctor to perform manual adjustments/stop adjustments, etc.)

Yet another adjustment protocol may include a slight deviation from the treatment plan by allowing one or more struts to adjust by a slight amount in a direction that may be counter to the direction prescribed in the treatment plan in order to offload an overloaded strut. By way of a non-limiting example, if one strut is determined to be overloaded and increasing the length of a strut adjacent to the overloaded strut increases a load experienced by the already overloaded strut, then the current subject matter may attempt to slightly shorten rather than lengthen the adjacent strut to offload the overloaded strut and then adjust all other struts according to the treatment plan. Such slight deviations from the treatment plan may be carefully controlled to ensure that patient's anatomy is not negatively affected at the expense of balancing strut loads. Also, deviations from the treatment plan are not limited to adjacent struts and/or single struts adjusting counter to the treatment plan to offload an overloaded strut.

In some examples, electronics of the spatial frame may be configured to incorporate various additional mechanisms that may be used to monitor and collect motor load data and/or determine strut loading and/or strut load balancing. By way of a non-limiting example, such mechanisms may include, strain gauges/sensors, force gauges/sensors, pressure gauges/sensors, and/or any other types of gauges/sensors that may be built into the strut assembly and/or frame assembly that may be configured to feed motor load/strut load/etc. data back to the system.

In some examples, upon detection of problematic strut load conditions (e.g., an overloaded strut), one or more of the adjustment protocols described above may be attempted to offload the strut(s). A first adjustment protocol may be selected and, subsequently, initiated. Then, the struts loads may be evaluated. If the strut load conditions have not sufficiently improved, then other adjustment protocols may be selected and initiated. If a predetermined number of adjustment protocols have been unsuccessful in offloading the overloaded strut(s), then the patient may be instructed to reduce the body weight applied to the spatial frame, e.g., if possible, by sitting, laying down, etc. to allow the overloaded strut to adjust according to the treatment plan. If the overloaded strut(s) cannot be sufficiently offloaded for normal adjustments to continue according to the treatment plan (e.g., at 718), then motorized adjustments may be halted and the patient may be instructed to consult their healthcare provider, at 720, as discussed above.

Using data that is fed back from a motorized strut in a hexapod to balance strut loading has various advantages over existing methods/systems (e.g., those that are used now with manual struts). For example, in conventional systems, uneven strut loading with manual struts is hard to detect, whereby the best indicators being struts that are difficult to adjust and/or patient pain. However, with current subject matter's ability to make changes to the adjustments of each strut to offload overloaded struts, ensure that treatment may continue without intervention while minimizing the risk of damage to the components of the motorized strut.

### B. Using Strut Motor Feedback to Determine Healing

As discussed above, use of hexapods provides an ability to treat deformities and/or traumatic injuries through limb alignment accomplished by adjusting six struts of the hexapod in accordance with a prescription or treatment plan. When limb alignment is completed, a patient typically stays in the spatial frame (e.g., shown in FIG. 1) until the injured bone(s) fully heals and hardens (consolidates). X-rays are taken periodically to assess bone healing; however, it is difficult to know when the bone is completely healed, and the frame can safely be removed.

For monitoring of consolidation status, after adjustments are completed, X-rays are taken regularly to assess multi-cortical bone healing. Typically, doctors/surgeons review at least three bone cortices making contact. However, with six struts in place, it can be difficult to observe the area of interest well without removing the frame. If the frame is removed too soon, the injured bone may not be healed enough to support the load of weight-bearing without the frame taking some load and thus, another fracture can occur. Conversely, since it is hard to tell when a patient is sufficiently healed, patients are often left in the frame longer than may be necessary to be on a safe side and avoid the frame being removed too soon.

In some cases, doctors/surgeons may check for bone healing by destabilizing the patient's frame. One way to destabilize the frame is to “unlock” one or more of its struts and have the patient bear weight on an unstable construct. Other healing-check methods include removing some fixation components (e.g., things like centering sleeves from rancho cubes). If the patient does not have pain when walking on a destabilized frame, then the doctors/surgeons may treat this as evidence that the frame may be removed. However, in some situations, this may be misleading as re-injury may occur, thereby forcing the patient to be placed back into the frame.

In some examples, the current subject matter may be configured to address the above issues and provide for monitoring of the bone healing process using some of the electronic components incorporated into the spatial frame. For example, the current subject matter may be configured to perform monitoring of healing by determining loading on each strut in the frame by way of receiving data related to battery current pull by a motor of each strut. The motor current pull may be determined during actuation of struts (one or more or all) in the frame. Data related to current pull may be gathered to determine strut loading, which in turn, may be used to assess bone healing/consolidation.

As discussed above, in some examples, the spatial frame may include a battery that may power the motor (either one motor coupled to all struts, a motor coupled to each strut, and/or a motor coupled to a group of struts), which, in turn, may be configured to operate the strut(s), such as, for example, to lengthen and/or shorten the strut(s). The strut operation may involve turning a gear that will interact with an internally threaded member that may be coupled to the threaded rod of the strut (as, for example, is shown in FIGS. 5A-B). As the strut is loaded with soft tissue loads and/or weight bearing loads, the amount of torque required from the motor to overcome the resistance in the system may vary. As the torque required varies, the current pulled from the battery by the motor (and/or each motor) may vary in a corresponding manner.

In some examples, motor load data may be used to determine how and/or whether the bone is healing. Once the adjustment phase of the treatment is complete, no further adjustments may need to be made by the motorized struts. However, one or more motor(s) may be activated and driven in one direction and then back in the other direction so that the length of each strut is not changed. In some examples, actuation of the motors may be for a very short period of time with the goal of lengthening the strut(s) as little as possible, e.g., long enough (e.g., tenths of a millimeter) to obtain a stable current load measurement from one or more motors. This may be performed periodically (e.g., once a day, a week, etc., and/or any other desired schedule) to obtain motor load data from each strut. In some examples, as the bone consolidates, the motor load may be configured to increase consistently, as it may take more force to distract a more fully healed bone. This correlation may be used to make a determination about bone's healing process. For example, the motor loading data that is gathered over time along with X-rays and/or clinical assessments may be used to make such determination.

In some examples, a machine-learning model may be developed and trained based on the historic motor loading data as related to a healing process of particular bone injuries to predict when the spatial frame may be safely removed. The development/training of machine-learning model may also rely on one or more of the following features: patient specific factors, such as, for example, age, weight, bone density, correction area (e.g., anatomy), deformity vs. trauma, comorbidities, etc. For instance, the relative change in motor load that may indicate full healing for a child may be different than for an adult. Once a specific user's information, as for example, related to the features above, is provided, the trained machine-learning model may be used to output an estimate time frame for removal of the spatial frame.

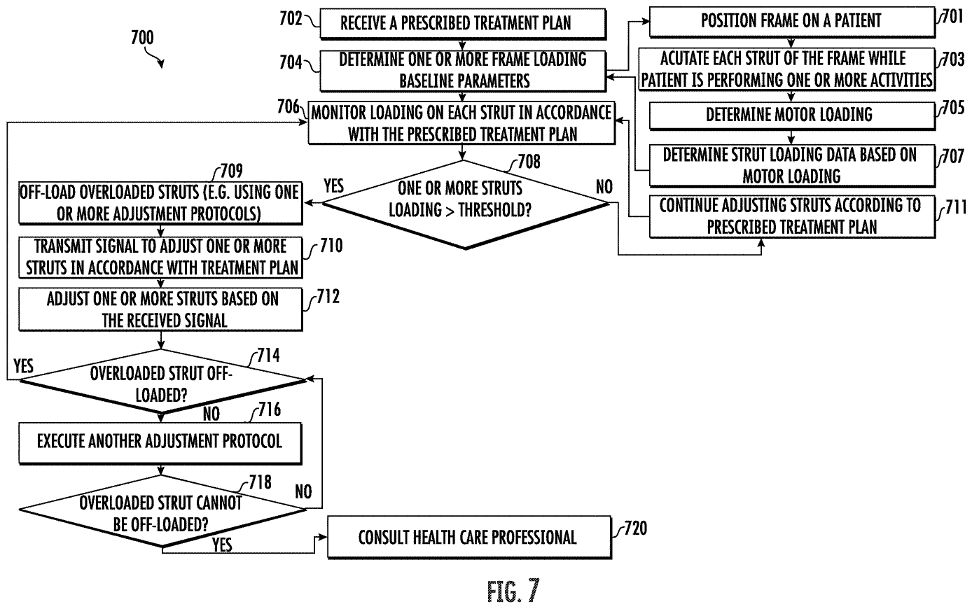

FIG. 8 illustrates an exemplary process 800 for executing a healing process assessment when using a spatial frame positioned on a patient, according to some examples of the current subject matter. The process 800 may be executed using a spatial frame shown in FIGS. 3a-6. In particular, the spatial frame may be communicatively coupled to one or more user devices (e.g., a mobile telephone, a tablet, a laptop, a personal computer, a personal digital assistant, and/or any other device) via one or more networks. The connection may be wireless and/or wired and/or any combination thereof.

Similar to the process 700 shown in FIG. 7, the spatial frame may be configured to incorporate one or more transceivers (e.g., wireless, wired, etc. transceivers) that may be configured to transmit and/or receive data from the spatial frame. The data may include current pull data corresponding to an amount of current drain from the battery and/or batteries that may be used as power source(s) by one or more motors used to perform movement of each and/or one or more motorized struts 106. The data may also include loading data (e.g., pressure, tension, etc.) exerted on each motorized strut 106 by the patient body, limbs, etc. during sitting, standing, performing one or more activities, etc.

Again, similar to the process 700, in some examples, the spatial frame may also incorporate one or more sensors that may be configured for measurement of strut loads, current pull loads, etc. By way of a non-limiting example, a sensor may be positioned on each strut for detecting strut load. Each such sensor may be configured to provide data to the transceiver for transmission to an external device for further processing (e.g., calculation of the load). Alternatively, or in addition, each such sensor may be configured to transmit its data directly to the external device. Further, a single sensor may be configured to monitor loading data for all struts individually and/or collectively and then provide its data to the frame's transceiver and/or directly to the external device. Alternatively, or in addition, a single sensor may be configured to monitor loading data for a group of struts, while other sensor(s) may be configured to monitor loading data for another group of struts, where each such sensor may be configured to report its monitored data to the transceiver and/or external device. The sensors may be configured to be communicatively coupled to one another and/or the transceiver and/or external device using a wireless and/or a wired connection and/or any combination thereof.

Alternatively, or in addition, the spatial frame may be configured to include processing (including machine-learning), storage, display, and/or communication capabilities for controlling actuation of each of the struts in the spatial frame, receiving/obtaining current loading data from the motor as a result of actuation of the struts and/or loading data from each of the struts, determining loading on each of the struts, and further determining bone healing status as a result of the determined loading. Such processing, storage, display and/or communication capabilities may be incorporated into the PCB of the spatial frame and/or be separately positioned anywhere on the frame.

At 802, a prescribed treatment plan associated with treatment of a patient using a spatial frame (e.g., shown in FIGS. 3a-6) may be received and applied (e.g., the spatial frame may be positioned on the patient). The treatment plan may be configured to indicate how the spatial frame needs to be positioned on the patient and/or how it needs to be adjusted during the course of the treatment and when.

At 804, one or more frame loading consolidation baseline parameters and/or a consolidation baseline may be established for the spatial frame at the beginning of bone consolidation process (in some instances, execution of the process 700 may have been completed at this time). To establish such baseline, at the beginning of the bone consolidation phase, the current subject matter may be configured to actuate each/some/all struts for a predetermined period of time while the patient is directed to stand, sit, walk and/or perform any other activity, at 803. At 805, motor loading data may be collected and/or determine in view of the activities. Using the motor loading data, the current subject matter may be configured to determine loading for each strut in the spatial frame, at 807. Again, this data may be used to establish the baseline thereby providing an understanding of the strut loads associated with soft tissue forces versus weight bearing forces at consolidation time.

In some examples, at 806, the current subject matter may also execute a machine-learning process to determine an expected bone healing time, and thus, potential time for removal of the spatial frame from the patient. Such process may be based on historical data, patient-related factors (e.g., age, weight, type of bone injury, soft tissues, etc.). The expected healing time may be compared against data obtained during monitoring of the healing of the bone to assess any deviations and/or for further training of machine-learning models involved in the machine learning process.

Once the baseline is established, the current subject matter may be configured to monitor for changes throughout the course of treatment, at 808. In some examples, the loading on each strut (and/or differences in loading vis-a-vie the loading baseline) may be monitored to ascertain whether the loading on each strut is within a predetermined healing range, which may be identified based on the determined baseline above, at 810. If no changes are detected, then the current subject matter may be configured to continue monitoring of loading on each strut of the spatial frame, at 808, and the frame may continue to remain on the patient.

However, if the current load pulled by the motors exceeds those established to be within the healing range (e.g., loading may be assessed for each strut, a group of struts, and/or all struts), the current subject matter may be configured to request the doctor/surgeon/etc. to confirm that the bone has fully healed, at 812. For example, the current subject matter may determine that the bone is healed when load on one or more or all struts is very low (or nothing), i.e., the bone is absorbing all the load. Alternatively, or in addition, the current subject matter may also determine that the bone is healed when high motor load associated with one or more or all struts'motors is detected. If healing is confirmed, the frame may be removed from the patient, at 814.

In some examples, electronics of the spatial frame may be configured to incorporate various additional mechanisms that may be used to monitor and collect motor load data to monitor/determine bone healing. By way of a non-limiting example, such mechanisms may include, strain gauges/sensors, force gauges/sensors, pressure gauges/sensors, and/or any other types of gauges/sensors that may be built into the strut assembly and/or frame assembly that may be configured to feed motor load/strut load/etc. data back to the system.

In some examples, the software components of the spatial frame may be configured to know one or more locations of points on the fixed (e.g., reference) and moving fragments. Such software may be programmed to actuate and control movement (at 803) of the struts in a plurality of directions. For example, direction and/or magnitude of motions may be assigned relative to the frame and/or to the bone segments.

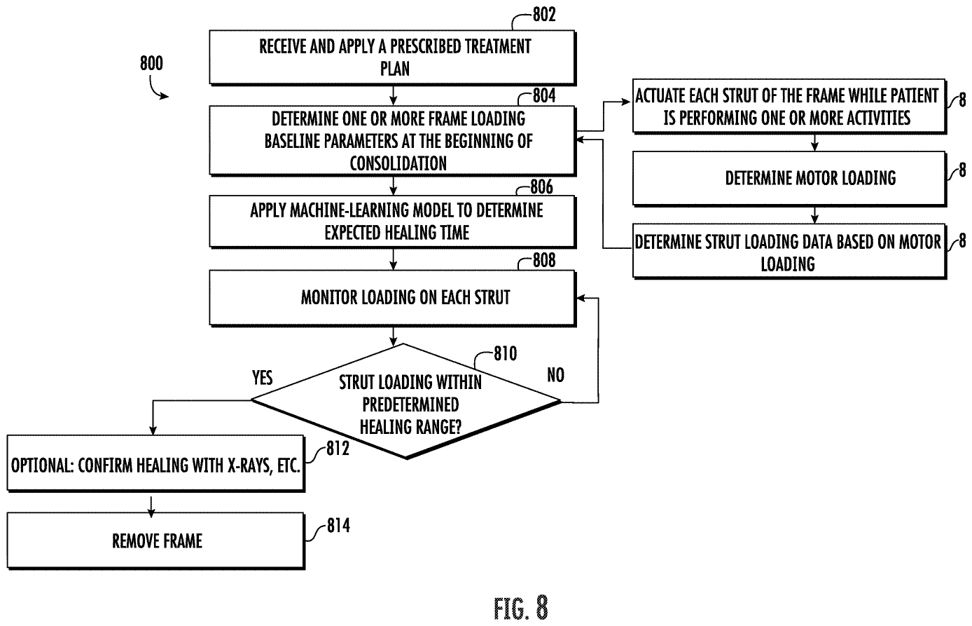

FIGS. 9A-C illustrate an exemplary movements of bone segments 902, according to some examples. In particular, the bone segments 902 may be configured to be moved in different directions for assessment of bone healing. As shown in FIGS. 9A-B, the motorized struts may be configured to translate bone segments in a plurality of directions. FIG. 9A illustrates exemplary axial translation motions 904, as shown by arrows “1”, “2” and “3”. The movement shown by arrow “1” illustrates compression motion for a predetermined distance; movement shown by arrow “2” illustrates distraction motion for a predetermined distance (which may be different from the compression motion distance); and movement shown by arrow “3” illustrates return to the original position (e.g., prior to movement shown by arrow “1”).

FIG. 9B illustrates exemplary non-axial translation motions 906, as shown by arrows “1”, “2” and “3”. The movement shown by arrow “1” illustrates motion in a first direction; movement shown by arrow “2” illustrates motion in an opposite direction; and movement shown by arrow “3” illustrates return to the original position (e.g., prior to movement shown by arrow “1”).

In some examples, a sequence of motions may be performed to assess healing in multiple directions since all cortices may not heal at the same rate. For example, the current subject matter may be configured to determine motor load while struts are being manipulated, such that the bone segments are translated to compress and/or distract, then translated anterior and/or posterior, and/or finally translated medial and/or then lateral. As can be understood, any combination, order, magnitude, and/or direction of motions is possible.

As shown in FIG. 9C, motorized struts may be actuated to manipulate bone segments to assess resistance to angulation motion 908, such as, for example, axial rotation, AP view angulation, LAT view angulation, and/or any bending motion. Similar to the translational motions shown in FIGS. 9A-B, angulation may be assessed in any direction, magnitude, sequence, and/or order.

As can be understood, motions inducing translation and/or angulation of the bone segments may be performed separately and/or as any combination of motions. In some examples, motor load limits may be implemented in the software of the spatial frame, such that, for example, manipulation of the struts will not occur if one or more predetermined thresholds are reached. This may apply to specific direction motions, motions within a sequence, and/or all motions. Further, the struts may also be programmed to manipulate the spatial frame in one or more predetermined directions on an increasing magnitude until motor load thresholds and/or a maximum safe magnitude for the motion(s) are reached. Motor load may be used to assess whether bone regenerate is forming as expected during adjustment according to the received prescription/treatment plan, not only after the adjustment phase is complete.

Advantageously, using load data from the frame to assess bone healing may provide doctors/surgeons with more information to make the best decision on when to remove the frame from a patient. The existing method of using radiographic evidence to determine healing visually is less than ideal, especially for doctors/surgeons with limited experience making such decisions. Visualizing the regenerate is not always easy and frame removal at the wrong time can lead to additional surgery for the patient, which the current subject matter method addresses.

### C. Using Strut Motor Feedback to Determine Adjustment Rate

In some examples, the current subject matter may be configured to use feedback data that it determines based on the motor current pull to determine an adjustment rate for one or more and/or all struts in the spatial frame. The adjustment rate of one or more/group/all struts may include at least one of the following components/parameters: a direction of adjustment, a length of adjustment, an angle of adjustment, a time for adjustment and/or any other parameters for changing positioning of each strut and hence, affecting the bone healing process.

As the bone segments move, the process of distraction osteogenesis takes place and bone is grown by the body to bridge the gap between the segments. One of the first decisions to be made by the doctor/surgeon when starting this process is how quickly the bone segments should be moved. Moving the segments 1 mm per day is often used as a starting point, but the health and age of the patient can drive a faster or slower rate. While using an adjustment rate of around 1 mm/day results in successful treatment for most patients, the ideal rate for each patient is dependent on a variety of factors that may be specific to that patient, bone injury, etc. Adjusting too quickly may negatively impact healing and adjusting too slowly may result in pre-consolidation of the patient's bone segments and revision surgery. Knowing the ideal rate for each patient and using that rate may result in better quality bone and less time in the frame.

Currently, the adjustment rate for each patient is determined by the doctor/surgeon supervising the treatment process. They make this decision based on the surgeon's experience and patient specific factors such as age, weight, fracture or deformity location, bone quality, etc. Once the initial rate is selected and adjustments begin, clinical follow up occurs weekly or bi-weekly for most patients and X-rays are taken to assess regenerate bone growth. Based on what is seen in the X-rays, the doctor/surgeon may choose to change the rate of adjustment at any point during treatment.

In some examples, motor current pull data associated with actuation of each/some/all of the struts may be used for the purposes of correlating how much current the motor pulls when making an adjustment with how much load each strut is observing. Based on this correlation, the adjustment rate(s) for each patient as well as how the rate may need to be changed during the course of treatment/healing may be determined.

Using motor load data that may be received from each motorized strut may be configured to be used to determine and/or maintain a particular adjustment rate for each patient/frame. The current subject matter may be configured to use changes in the motor load associated with one or more struts to determine whether adjustments/movements of such strut(s) are being performed either too quickly and/or too slowly. For instance, if motor loads are increasing significantly as the treatment progresses, the current subject matter may be configured to determine that the adjustment rate is too slow, thereby causing the bone to be pulled apart that has undergone more healing than is optimal. Conversely, if motor loads are decreasing substantially, the current subject matter may be configured to determine that the adjustment rate is being performed too fast.

In some examples, the current subject matter may be configured to account for various confounding variables, such as, for example, increasing soft tissue forces over the course of treatment, patient anatomy, specific bone injury being treated, etc. In some examples, a machine-learning model may be developed and trained based on the historic motor loading data as related to a healing process of particular bone injuries to predict specific adjustment rate and/or any changes of adjustment rates. The development/training of such machine-learning model may rely on one or more of the following features: patient specific factors, e.g., age, weight, bone density, correction area (anatomy), deformity vs. trauma, comorbidities, etc. For instance, an ideal adjustment rate for a child is going to be higher than for an adult because children can grow bone faster than adults (in general). Once a specific user's information, as for example, related to the features above, is provided, the trained machine-learning model may be used to output a particular adjustment rate and/or changes to adjustment rate throughout the course of treatment. Further, using motor load data, the current subject matter may be configured to determine how the load through the frame may be changing throughout the treatment and modulate the adjustment rate as needed to maintain consistent motor loading.

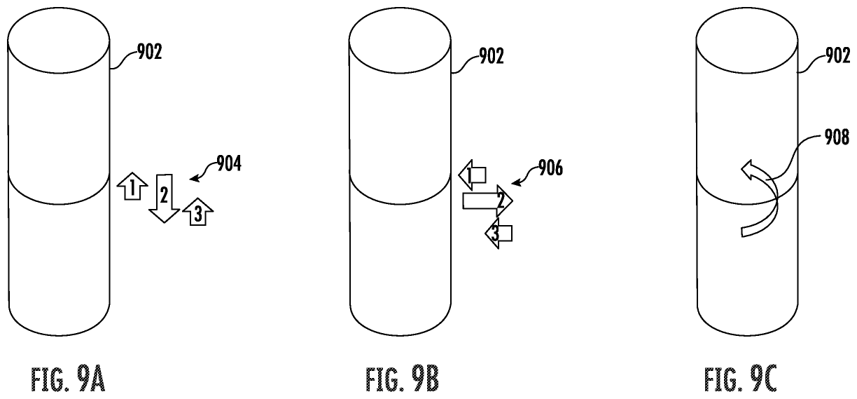

FIG. 10 illustrates an exemplary process 1000 for determining and modifying an adjustment rate of one or more struts in a spatial frame positioned on a patient, according to some examples of the current subject matter. The process 1000 may be executed using a spatial frame shown in FIGS. 3a-6. In particular, the spatial frame may be communicatively coupled to one or more user devices (e.g., a mobile telephone, a tablet, a laptop, a personal computer, a personal digital assistant, and/or any other device) via one or more networks. The connection may be wireless and/or wired and/or any combination thereof.

Similar to the processes 700 and 800 discussed above, the spatial frame may be configured to incorporate one or more transceivers (e.g., wireless, wired, etc. transceivers) that may be configured to transmit and/or receive data from the spatial frame. The data may include current pull data corresponding to an amount of current drain from the battery and/or batteries that may be used as power source(s) by one or more motors used to perform movement of each and/or one or more motorized struts 106. The data may also include loading data (e.g., pressure, tension, etc.) exerted on each motorized strut 106 by the patient body, limbs, etc. during sitting, standing, performing one or more activities, etc.

As discussed above, the spatial frame may also incorporate one or more sensors that may be configured for measurement of strut loads, current pull loads, etc., where sensor(s) may be configured to send data to the transceiver for transmission to an external device for further processing (e.g., calculation of the load) and/or to transmit its data directly to the external device. The sensors may be configured to be communicatively coupled to one another and/or the transceiver and/or external device using a wireless and/or a wired connection and/or any combination thereof. Alternatively, or in addition, the spatial frame may be configured to include processing, storage and/or communication capabilities for controlling actuation of each of the struts in the spatial frame, receiving/obtaining current loading data from the motor as a result of actuation of the struts and/or loading data from each of the struts, determining loading on each of the struts, determining adjustment rates and/or modifying adjustment rates, and further controlling adjustments of each strut as a result of the determined/modified adjustment rates. Such processing, storage and/or communication capabilities may be incorporated into the PCB of the spatial frame and/or be separately positioned anywhere on the frame.

At 1002, a prescribed treatment plan associated with treatment of a patient using a spatial frame (e.g., shown in FIGS. 3a-6) may be received. At 1004, one or more frame loading baseline parameters and/or a baseline may be established for the spatial frame in accordance with the plan. The spatial frame may be positioned on the patient (e.g., the frame may be positioned on the patient during a surgical procedure, whereby the motors may already be included in the frame and/or coupled to the respective struts at another time), at 1001, each/some/all struts may be actuated for a predetermined period of time while the patient is stands, sits, walks and/or performs any other activity, at 1003, and motor loading data in the spatial frame may be obtained/collected, at 1005, thereby indicating loading of the struts, at 1007, in view of such activities. Similar to processes 700 and 800 above, such data may be used to determine the loading baseline (e.g., the strut loads associated with soft tissue forces versus weight bearing forces).

Once the loading baseline is established, the current subject matter may be configured to determining one or more adjustment rates, at 1006. Such determination may be made using, for example, a machine-learning model, which may be queried using patient-specific parameters (e.g., age, weight, height, type of injury, etc.), type of spatial frame, and/or any other variables. The rate and/or multiple rates may be determined and may be indicative of a specific direction of adjustment (e.g., linear, angular, etc.), type of adjustment (e.g., translation, rotation, etc.), speed of adjustment, length/distance of adjustment monitor, time when adjustment needs to be made, and/or any other parameters. The rate(s) of adjustment may be determined for each strut, group of struts, and/or all struts.

In some examples, the loading on each strut (and/or differences in loading vis-a-vie the loading baseline) may be monitored, at 1008. The monitoring may be made in view of various thresholds that may be established by the treatment plan. If no changes are detected, at 1010, then the current subject matter may be configured to continue monitoring of loading on each strut of the spatial frame, at 1008.

However, if the loading on one or more struts exceeds a predetermined threshold (e.g., a significant change in the strut loading is detected), the current subject matter may be configured to execute a modification of the determined adjustment rate, at 1012 to ensure the no strut(s) is overloaded. As a result of modifying of the adjustment rate, the current subject matter may be configured to change the specific direction of adjustment (e.g., linear, angular, etc.), the type of adjustment (e.g., translation, rotation, etc.), the speed of adjustment, the length/distance of adjustment monitor, time when adjustment needs to be made, and/or any other parameters for one or more struts and/or group of struts and/or all struts.

In some examples, and similar to the discussion above, electronics of the spatial frame may incorporate various components that may monitor and/or collect motor load data and/or determine strut loading and/or strut load balancing, such as, for example, strain gauges/sensors, force gauges/sensors, pressure gauges/sensors, and/or any other types of gauges/sensors. These components along with various processing components may be used to determine an ideal adjustment rate and/or modify such rate as needed. In some examples, the spatial frame processors may be programmed (e.g., automatically, by a doctor/surgeon, etc.) with various upper and/or lower adjustment rate limits. The spatial frame may then use such limits to speed up and/or slow down adjustments in the adjustment phase of treatment according to motor load data as long as the rate of adjustment remains within the set limits. If motor load data indicates that the limits are still interfering with treatment, adjustments may be terminated and/or re-evaluated by the doctor/surgeon.

Using data that is fed back from a motorized strut in a hexapod to determine an adjustment rate is advantageous over existing methods, which involve frequently taking X-rays (e.g., every week and/or every two weeks) throughout treatment and evaluating the regenerate bone visually. Such methods are inconsistent/inadequate means for determining whether the selected adjustment rate is optimal, especially for surgeons with less experience making this assessment.

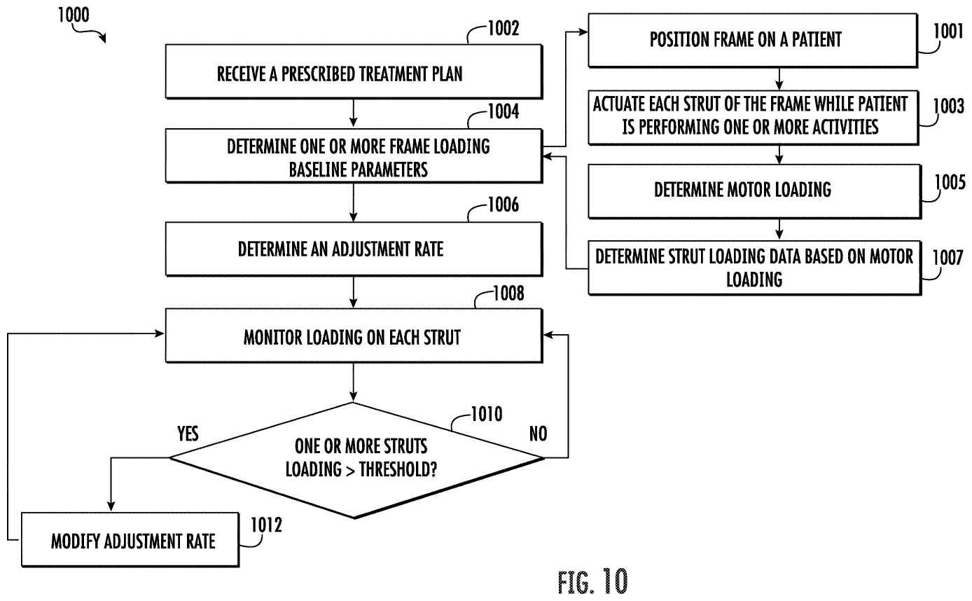

FIG. 11 illustrates an exemplary computing apparatus 1100, according to some examples of the current subject matter. The apparatus 1100 may be a computing device that may be communicatively coupled with a spatial frame and/or device communicatively coupled to the spatial frame such as, spatial frame shown in FIGS. 3a-6. The apparatus 1100 may be a computer in the form of a smart phone, a tablet, a notebook, a desktop computer, a workstation, or a server. The apparatus 1100 can combine with any suitable example of the systems, devices, and methods disclosed herein. The apparatus 1100 can include processor(s) 1110, a non-transitory storage medium 1120, communication interface 1130, and a display 1135. The processor(s) 1110 may comprise one or more processors, such as a programmable processor (e.g., a central processing unit (CPU)). The processor(s) 1110 may comprise processing circuitry to implement spatial frame circuitry 1115.

The processor(s) 1110 may include memory such as flash memory to contain program code for execution by the processor(s) 1110. In some implementations, the processor(s) 1110 may have random access memory to contain a copy of code from flash memory or read only memory to facilitate faster execution of code. In some implementations, the processor(s) 1110 may include cache to contain data for faster calculations or execution. In some implementations, the processor(s) 1110 may include spatial frame circuitry 1115, which may include a user interface manager 1117. The user interface manager 1117 may function as a state machine controlled by keypad inputs, internal events or alarms, boundary conditions, exceptions, and supervisory input to the user interface manager 1117. The user interface manager 1117 may process button presses and may update a main screen on the display 1135 reflecting the state of the application.

Motor controller commands may be executed automatically and/or upon the user's actions via button presses, system states, and error conditions. Further, the user interface manager 1117 may implement alerts, warnings, and notifications and display the alerts, warnings, and notifications via the display 1135. The user interface manager 1117 may also include code to handle the user's response to alerts, warnings, and notifications.

The processor(s) 1110 may operatively couple with a non-transitory storage medium 1120. The non-transitory storage medium 1120 may store logic, code, and/or program instructions executable by the processor(s) 1110 for performing one or more instructions including the spatial frame circuitry 1125. The non-transitory storage medium 1120 may include one or more memory units (e.g., fixed and/or removable media or external storage such as electrically erasable programmable read only memory (EEPROM), a secure digital (SD) card, random-access memory (RAM), a flash drive, solid-state drive, a hard drive, and/or the like). The memory units of the non-transitory storage medium 1120 may store logic, code and/or program instructions executable by the processor(s) 1110 to perform any suitable implementation of the methods described herein. For example, the processor(s) 1110 may execute instructions such as instructions of spatial frame circuitry 1125 causing one or more processors of the processor(s) 1110 to communicate user commands to the spatial frame 300 (as shown in FIGS. 3a-6) and/or to communicate events, alerts, operation parameters for the spatial frame 300, and configurations.

The panels 1128 may define graphical user interfaces for display of information and for receiving input parameters or configurations from a user. The configuration file 1129 may include user selected parameters such as, for example, adjustment rates, motor pull, current pull, etc.

The processor(s) 1110 may couple to a communication interface 1130 to transmit the data, code, or commands to and/or receive data, code, or commands from one or more external devices (e.g., a terminal, display device, a smart phone, a tablet, a server, or other remote device). The communication interface 1130 includes circuitry to transmit and receive communications through a wired and/or wireless media such as an Ethernet interface, a wireless fidelity (Wi-Fi) interface, a Bluetooth interface such as a Bluetooth Low Energy (BLE) interface, a cellular data interface, and/or the like. In some examples, the communication interface 1130 may implement logic such as code in a baseband processor to interact with a physical layer device to transmit and receive wireless communications from the spatial frame 300. For example, the communication interface 1130 may implement one or more of local area networks (LAN), wide area networks (WAN), infrared, radio, Bluetooth, Wi-Fi, point-to-point (P2P) networks, telecommunication networks, cloud communication, and the like.

The processor(s) 1110 may couple to a display 1135 to display panels 1128 for a user interface and/or other user interface items such as a message or notification via, graphics, video, text, and/or the like. In some examples, the display 1135 may include a display on a terminal, a display device, a smart phone, a tablet, a server, or a remote device.

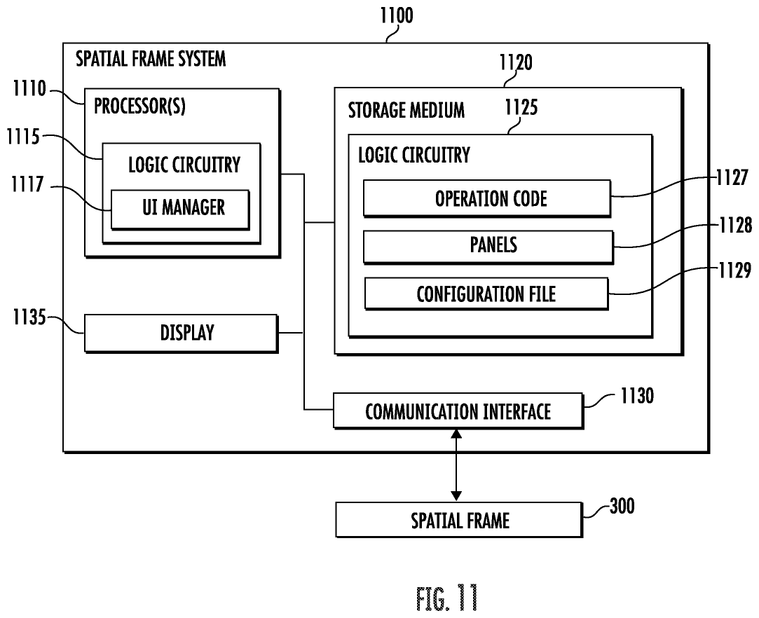

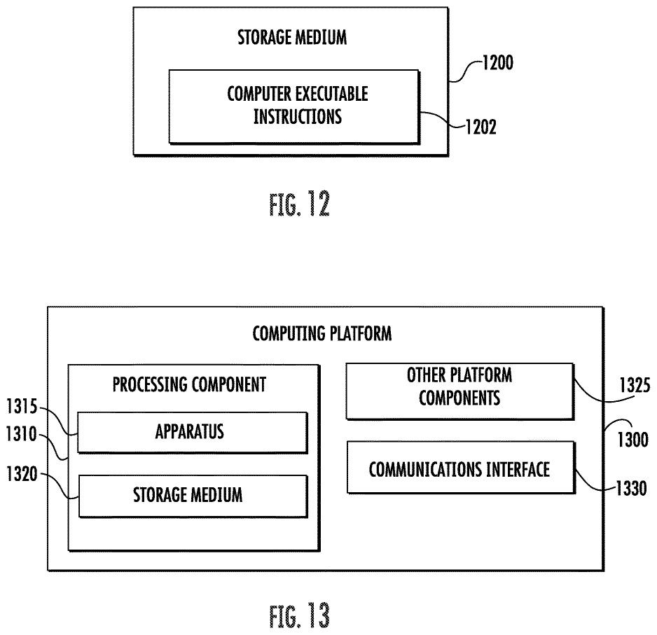

FIGS. 12-13 illustrate example implementations of a storage medium and computing platform for a spatial frame in accordance with one or more features of the present disclosure. FIG. 12 illustrates an example of a storage medium 1200 to store spatial frame logic. Storage medium 1200 may include an article of manufacture. In some examples, storage medium 1200 may include any non-transitory computer readable medium or machine-readable medium, such as an optical, magnetic or semiconductor storage. Storage medium 1200 may store various types of computer executable instructions 1202, such as instructions to implement logic flows and/or techniques described herein. Examples of a computer readable or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions may include any suitable type of code, such as source code, compiled code, interpreted code, executable code, static code, dynamic code, object-oriented code, visual code, and the like. The examples are not limited in this context.

FIG. 13 illustrates an example computing platform 1300. In some examples, as shown in FIG. 13, the computing platform 1300 may include a processing component 1310, other platform components or a communications interface 1330. According to some examples, computing platform 1300 may be implemented in a computing device such as a server in a system such as a data center or server farm that supports a manager or controller for managing configurable computing resources as mentioned above. Further, the communications interface 1330 may include a wake-up radio (WUR) and may be capable of waking up a main radio of the computing platform 1300.

According to some examples, processing component 1310 may execute processing operations or logic for apparatus 1315 described herein such as the spatial frame logic circuitry 1115, and 1125 illustrated in FIG. 11. Processing component 1310 may include various hardware elements, software elements, or a combination of both. Examples of hardware elements may include devices, logic devices, components, processors, microprocessors, circuits, processor circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), memory units, logic gates, registers, semiconductor device, chips, microchips, chip sets, and so forth. Examples of software elements, which may reside in the storage medium 1320, may include software components, programs, applications, computer programs, application programs, device drivers, system programs, software development programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an example is implemented using hardware elements and/or software elements may vary in accordance with any number of factors, such as desired computational rate, power levels, heat tolerances, processing cycle budget, input data rates, output data rates, memory resources, data bus speeds and other design or performance constraints, as desired for a given example.

In some examples, other platform components 1325 may include common computing elements, such as one or more processors, multi-core processors, co-processors, memory units, chipsets, controllers, peripherals, interfaces, oscillators, timing devices, video cards, audio cards, multimedia input/output (I/O) components (e.g., digital displays), power supplies, and so forth. Examples of memory units may include without limitation various types of computer readable and machine readable storage media in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (e.g., USB memory), solid state drives (SSD) and any other type of storage media suitable for storing information.

In some examples, communications interface 1330 may include logic and/or features to support a communication interface. For these examples, communications interface 1330 may include one or more communication interfaces that operate according to various communication protocols or standards to communicate over direct or network communication links. Direct communications may occur via use of communication protocols or standards described in one or more industry standards (including progenies and variants) such as those associated with the PCI Express specification. Network communications may occur via use of communication protocols or standards such as those described in one or more Ethernet standards promulgated by the Institute of Electrical and Electronics Engineers (IEEE). For example, one such Ethernet standard may include IEEE 802.3-2012, Carrier sense Multiple access with Collision Detection (CSMA/CD) Access Method and Physical Layer Specifications, Published in December 2012. Network communication may also occur according to one or more OpenFlow specifications such as the OpenFlow Hardware Abstraction API Specification. Network communications may also occur according to InfiniBand Architecture Specification, Volume 1, Release 1.3, published in March 2015.

Computing platform 1300 may be part of a computing device that may be, for example, a server, a server array or server farm, a web server, a network server, an Internet server, a workstation, a mini-computer, a main frame computer, a supercomputer, a network appliance, a web appliance, a distributed computing system, multiprocessor systems, processor-based systems, or combination thereof. Accordingly, functions and/or specific configurations of computing platform 1300 described herein, may be included, or omitted in various implementations of computing platform 1300, as suitably desired.

The components and features of computing platform 1300 may be implemented using any combination of discrete circuitry, ASICs, logic gates and/or single chip architectures. Further, the features of computing platform 1300 may be implemented using microcontrollers, programmable logic arrays and/or microprocessors or any combination of the foregoing where suitably appropriate. It is noted that hardware, firmware and/or software elements may be collectively or individually referred to herein as “logic.”

It should be appreciated that the exemplary computing platform 1300 shown in the block diagram of FIG. 13 may represent one functionally descriptive example of many potential implementations. Accordingly, division, omission or inclusion of block functions depicted in the accompanying figures does not infer that the hardware components, circuits, software and/or elements for implementing these functions would necessarily be divided, omitted, or included in implementations.

One or more features of at least one example may be implemented by representative instructions stored on at least one machine-readable medium which represents various logic within the processor, which when read by a machine, computing device or system causes the machine, computing device or system to fabricate logic to perform the techniques described herein. Such representations, known as “IP cores,” may be stored on a tangible, machine readable medium and supplied to various customers or manufacturing facilities to load into the fabrication machines that actually make the logic or processor.

The foregoing description has broad application. While the present disclosure refers to certain implementations, numerous modifications, alterations, and changes to the described implementations are possible without departing from the sphere and scope of the present disclosure, as defined in the appended claim(s). Accordingly, it is intended that the present disclosure not be limited to the described implementations. Rather these implementations should be considered as illustrative and not restrictive in character. All changes and modifications that come within the spirit of the current subject matter are to be considered within the scope of the disclosure. The present disclosure should be given the full scope defined by the language of the following claims, and equivalents thereof. The discussion of any implementation is meant only to be explanatory and is not intended to suggest that the scope of the disclosure, including the claims, is limited to these implementations. In other words, while illustrative implementations of the disclosure have been described in detail herein, it is to be understood that the inventive concepts may be otherwise variously embodied and employed, and that the appended claims are intended to be construed to include such variations, except as limited by the prior art. Unless otherwise defined, all technical terms used herein have the same meaning as commonly understood by one of ordinary skill in the art to which the disclosure belongs.

Directional terms such as top, bottom, superior, inferior, medial, lateral, anterior, posterior, proximal, distal, upper, lower, upward, downward, left, right, longitudinal, front, back, above, below, vertical, horizontal, radial, axial, clockwise, and counter-clockwise) and the like may have been used herein. Such directional references are only used for identification purposes to aid the reader's understanding of the present disclosure. For example, the term “distal” may refer to the end farthest away from the medical professional/operator when introducing a device into a patient, while the term “proximal” may refer to the end closest to the medical professional when introducing a device into a patient. Such directional references do not necessarily create limitations, particularly as to the position, orientation, or use of this disclosure. As such, directional references should not be limited to specific coordinate orientations, distances, or sizes, but are used to describe relative positions referencing particular implementations. Such terms are not generally limiting to the scope of the claims made herein. Any implementation or feature of any section, portion, or any other component shown or particularly described in relation to various implementations of similar sections, portions, or components herein may be interchangeably applied to any other similar implementation or feature shown or described herein.

It should be understood that, as described herein, an “implementation” (such as illustrated in the accompanying Figures) may refer to an illustrative representation of an environment or article or component in which a disclosed concept or feature may be provided or embodied, or to the representation of a manner in which just the concept or feature may be provided or embodied. However, such illustrated implementations are to be understood as examples (unless otherwise stated), and other manners of embodying the described concepts or features, such as may be understood by one of ordinary skill in the art upon learning the concepts or features from the present disclosure, are within the scope of the disclosure. Furthermore, references to “one implementation” of the present disclosure are not intended to be interpreted as excluding the existence of additional implementations that also incorporate the recited features.

In addition, it will be appreciated that while the Figures may show one or more implementations of concepts or features together in a single implementation of an environment, article, or component incorporating such concepts or features, such concepts or features are to be understood (unless otherwise specified) as independent of and separate from one another and are shown together for the sake of convenience and without intent to limit to being present or used together. For instance, features illustrated or described as part of one implementation can be used separately, or with another implementation to yield a still further implementation. Thus, it is intended that the present subject matter covers such modifications and variations as come within the scope of the appended claims and their equivalents.

As used herein, an element or step recited in the singular and proceeded with the word “a” or “an” should be understood as not excluding plural elements or steps, unless such exclusion is explicitly recited. It will be further understood that the terms “includes” and/or “comprising,” or “includes” and/or “including” when used herein, specify the presence of stated features, regions, steps, elements and/or components, but do not preclude the presence or addition of one or more other features, regions, integers, steps, operations, elements, components and/or groups thereof.

The phrases “at least one,” “one or more,” and “and/or” as used herein, are open-ended expressions that are both conjunctive and disjunctive in operation. The terms “a” (or “an”), “one or more”and “at least one”can be used interchangeably herein.

Connection references (e.g., engaged, attached, coupled, connected, and joined) are to be construed broadly and may include intermediate members between a collection of elements and relative to movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another. The drawings are for purposes of illustration only and the dimensions, positions, order and relative to sizes reflected in the drawings attached hereto may vary.

The foregoing discussion has been presented for purposes of illustration and description and is not intended to limit the disclosure to the form or forms disclosed herein. For example, various features of the disclosure are grouped together in one or more implementations or configurations for the purpose of streamlining the disclosure. However, it should be understood that various features of the certain implementations or configurations of the disclosure may be combined in alternate implementations or configurations. Moreover, the following claims are hereby incorporated into this Detailed Description by this reference, with each claim standing on its own as a separate implementation of the present disclosure.

1. A spatial frame apparatus, comprising:
a frame configured to support one or more motorized struts, wherein the one or more motorized struts comprise one or more motors configured to cause movement of the one or more motorized struts in one or more directions in accordance with one or more treatment plans;
one or more processing components communicatively coupled to at least one of: the one or more motorized struts and the one or more motors;
wherein the one or more processing components are configured to determine one or more loading parameters on the one or more motorized struts;
wherein the one or more loading parameters define loading on the one or more motorized struts upon the frame being at least one of: positioned on a patient and adjusted on the patient; and
wherein the one or more processing components execute at least one adjustment in one or more adjustments of the one or more motorized struts based on the one or more loading parameters.

2. The apparatus of claim 1, further comprising one or more monitoring components selected from one of the following: one or more sensors, one or more gauges, and any combination thereof, the one or more monitoring components configured to perform monitoring of at least one of the following: loading on the one or more motorized struts, current pull by the one or more motors from one or more power sources communicatively coupled to the one or more motors, and any combination thereof.

3. (canceled)

4. The apparatus of claim 1, wherein the one or more processing components determine one or more baseline loading parameters based on at least one action performed by the patient after the frame has been applied to the patient;
wherein the one or more baseline loading parameters define one or more initial loading parameters exerted on the frame and/or one or more motorized struts while the frame is positioned on the patient during performance of the action; and
wherein the one or more loading parameters include the one or more baseline loading parameters.

5. (canceled)

6. (canceled)

7. The apparatus of claim 1, wherein one or more loading thresholds, defined for the one or more motorized struts, indicate a predetermined amount of loading exerted by the patient on the frame and/or one or more motorized struts while the frame is positioned on the patient during a treatment defined in the one or more treatment plans.

8. The apparatus of claim 7, wherein upon determining that loading on the one or more motorized struts exceeds the one or more loading thresholds:
the one or more motors cause the one or more motorized struts to execute one or more adjustments; and/or
one or more communication components, communicatively coupled to the one or more processing components, transmit one or more instructions to the one or more processing components to cause execution of the one or more adjustments.

9. The apparatus of claim 8, wherein the one or more adjustments are defined by at least one of the following: a direction of the one or more adjustments of the one or more motorized struts, a length of the one or more adjustments of the one or more motorized struts, an angle of the one or more adjustments of the one or more motorized struts, a time for the one or more adjustments of the one or more motorized struts, a change in positioning of the one or more motorized struts, and any combination thereof.

10. (canceled)

11. The apparatus of claim 1, further comprising one or more machine-learning components, the one or more machine-learning components determine an expected healing time associated with a treatment of an injury to the patient.

12. The apparatus of claim 11, wherein the one or more machine-learning components include one or more machine-learning models trained using at least one of the following: one or more historic motor loading data associated with a healing process of injuries, one or more patient specific factors, an age of the patient, a weight of the patient, a bone density of the patient, an anatomical correction area of the patient, a deformity of the patient, a patient's trauma, one or more comorbidities of the patient, and any combination thereof.

13. The apparatus of claim 12, wherein the one or more processing components, using the one or more machine-learning models, predict a time for completion of the healing process and removal of the frame from the patient.

14. The apparatus of claim 12, wherein the one or more processing components, using the one or more machine-learning components, determine one or more adjustment rates for adjusting a movement of the one or more motorized struts during a treatment defined in the one or more treatment plans.

15. The apparatus of claim 12, wherein the one or more processing components, using the one or more machine-learning components, predict at least one of: one or more adjustment rates and one or more changes to the one or more adjustment rates.

16. The apparatus of claim 14, wherein each adjustment rate in the one or more adjustment rates is associated with a predetermined adjustment rate threshold.

17. The apparatus of claim 16, wherein the one or more processing components modify the one or more adjustment rates based on a determination that at least one adjustment rate in the one or more adjustment rates exceeds the predetermined adjustment rate threshold associated with the at least one adjustment rate as a result of one or more adjustments performed by the one or more motorized struts.

18. A method, comprising:
receiving, using one or more processing components, one or more treatment plans for a spatial frame apparatus, the spatial frame apparatus including:
a frame configured to support one or more motorized struts, wherein the one or more motorized struts comprise one or more motors configured to cause movement of the one or more motorized struts in one or more directions in accordance with the one or more treatment plans;
the one or more processing components communicatively coupled to at least one of: the one or more motorized struts and the one or more motors;
determining, using the one or more processing components, one or more loading parameters on the one or more motorized struts, wherein the one or more loading parameters define loading on the one or more motorized struts upon the frame being at least one of: positioned on a patient and adjusted on the patient; and
executing, using the one or more processing components, at least one adjustment in one or more adjustments of the one or more motorized struts based on the one or more loading parameters.

19. The method of claim 18, further comprising:
monitoring, using one or more monitoring components, at least one of the following: loading on the one or more motorized struts, current pull by the one or more motors from one or more power sources communicatively coupled to the one or more motors, and any combination thereof.

20. (canceled)

21. The method of claim 18, further comprising:
determining, using the one or more processing components, one or more baseline loading parameters based on at least one action performed by the patient after the frame has been applied to the patient;
wherein the one or more baseline loading parameters define one or more initial loading parameters exerted on the frame and/or one or more motorized struts while the frame is positioned on the patient during performance of the action; and
wherein the one or more loading parameters include the one or more baseline loading parameters.

22. (canceled)

23. (canceled)

24. The method of claim 18, wherein one or more loading thresholds, defined for the one or more motorized struts, indicate a predetermined amount of loading exerted by the patient on the frame and/or one or more motorized struts while the frame is positioned on the patient during a treatment defined in the one or more treatment plans.

25. The method of claim 24, wherein upon determining that loading on the one or more motorized struts exceeds the one or more loading thresholds:
the one or more motors cause the one or more motorized struts to execute one or more adjustments, the one or more adjustments are defined by at least one of the following: a direction of the one or more adjustments of the one or more motorized struts, a length of the one or more adjustments of the one or more motorized struts, an angle of the one or more adjustments of the one or more motorized struts, a time for the one or more adjustments of the one or more motorized struts, a change in positioning of the one or more motorized struts, and any combination thereof; and/or
one or more communication components, communicatively coupled to the one or more processing components, transmit one or more instructions to the one or more processing components to cause execution of the one or more adjustments.

26. (canceled)

27. (canceled)

28. The method of claim 18, further comprising determining, using one or more machine-learning components executed by the one or more processing components, determine an expected healing time associated with a treatment of an injury to the patient.

29. The method of claim 28, wherein the one or more machine-learning components include one or more machine-learning models trained using at least one of the following: one or more historic motor loading data associated with a healing process of injuries, one or more patient specific factors, an age of the patient, a weight of the patient, a bone density of the patient, an anatomical correction area of the patient, a deformity of the patient, a patient's trauma, one or more comorbidities of the patient, and any combination thereof.

30. The method of claim 29, further comprising predicting, using the one or more processing components, using the one or more machine-learning models, a time for completion of the healing process and removal of the frame from the patient.

31. The method of claim 29, further comprising determining, using the one or more processing components, using the one or more machine-learning components, one or more adjustment rates for adjusting a movement of the one or more motorized struts during a treatment defined in the one or more treatment plans.

32. The method of claim 29, further comprising predicting, using the one or more processing components, using the one or more machine-learning components, at least one of: one or more adjustment rates and one or more changes to the one or more adjustment rates.

33. The method of claim 31, wherein each adjustment rate in the one or more adjustment rates is associated with a predetermined adjustment rate threshold.

34. The method of claim 33, further comprising modifying, using the one or more processing components, the one or more adjustment rates based on a determination that at least one adjustment rate in the one or more adjustment rates exceeds the predetermined adjustment rate threshold associated with the at least one adjustment rate as a result of one or more adjustments performed by the one or more motorized struts.
